SEQUENCE LISTING (25 sequences)

SEQ ID NO: 1 - protein, 95 aa, unidentified
  source 1..95: note = Pichinde virus
MGLRYSKEVRKRHGDEDVVGRVPMTLNLPQGLYGRFNCKSCWFVNKGLIRCKDHYLCLGCLTKMHSRGNLCEICGHSLPTKMEFLESPSAPPYEP

SEQ ID NO: 2 - protein, 2194 aa, unidentified
  source 1..2194: note = Pichinde virus
MEEYVFELKDIVRKWVPEWEELSEQKNNVLAQVKDRAITIEGLKLLSMLVEVDSCKKHSCKHNTKMTVNAILRELRVTCPTLPDVTPDGYCMVGDVLILLEVFVRTSQEAFEKKYNQDFLKLLQLSSDLKRQNITLVPVIDGRSSYYVEFVPDWVVERLRWLLLKLMDGLRTSGEEVEELEYERLISSLSSLENQSLGLESLLAVKERGLPYKVRLEKALMSGINNKLTTDQCRTKIMEIFQQFKMLQLAGQLDRKLQATDREDMISRLQNHEFIQCSVKDVPKSEIRLCEFCSVHILGIIGQLRQSEVKHSSTESREYFRVLSICNKIKSQKVFNTRRNTMLVLDLIMYNILCDLDKSSPGAVFREVLLMQGLPSVNDRLINVDFLMEQITKKFIKNPNWLEKAKKRLSSVCGELPLDDILPLLREPDVEYYFNLKTSVLDEWGAKPCLQYKTKSQCMCGGRPGRGQPDYTIMGESEFEELLKTLSSLSLSLINSMKTAAVPKMKVNNADEFYGKVYCDEVFFQRFGEGGSLTLLYQKTGERSRCYAVAYRSKSGGLYETKASFYCDPKRFFLPIFSADVIQRTCVEMLSWLDFMSQPLLDSVSDLLRRLILCILCTPSKRIQVYLQGFRYYIMAFVNEVHFKELFEKLKVVMLTPSEWQTAMLIDDLILLVLSNSREEDMAKIFKFVLNVSYLCHFITKETPDRLTDQIKCFEKFLEPKLKFDSVLVNPSNSMELPTEEEEKMVHDIERLLGKKLESKCEGRPGLNKDVLSVCLSLFNSSSLEVKPLLPCDPMTPSFTSTALDMSSNKSVVVPKLNEVGEVITEYDYSSIVSAVVVEMIEHFKTKGKYKLDPKEVNFKILKRLSSLIQIKKESIEPDGVEELLSEDQGDCLKEIETRVAKVLSKVDTNVKTNLKTSCPLERLWPKSTMVVIKRETSLHDVKDFDYSLFSAEVYEDLVNLIYEDVTARSVYFADRLMNPCPLEFLIKNLTLKAYKEADYFECFKYILIASDYDNRVGRYDHKSRSRLGFTDAALQIRETSRISSRESNSESIAKRLDQSFFTNSSLRNLCFYSDESPTERSGVSTNVGRLKFGLSYKEQVGGNRELYVGDLNTKLTTRLIEDYSESLMQNMRYTCLNNEKEFERALLDMKSVVRQSGLAVSMDHSKWGPHMSPVIFAALLKGLEFKLKDGSEVPNAAVINILLWHIHKMVEVPFNVVEAYMKGFLKRGLGMMDKGGCTIAEEFMFGYFEKGKVPSHISSVLDMGQGILHNTSDLYGLITEQFINYALELCYGARFISYTSSDDEIMLSLNEGFKFKDRDELNVELVLDCMEFHYFLSDKLNKFVSPKTVVGTFASEFKSRFFIWSQEVPLLTKFVAAALHNIKAKAPNQQADTIDTILDQCVANGVSIEVVGAIAKRTNSMIIYSGFPNDPFLCLEEMDVLDWVNGSRGYRLQRSIETLFPDDLLLSIIRKACRKIFYKIQSGALEESYIVTTLQQSPDDCLKQLLETCDVETEAIEDALNIRWLNLRVHGDLRLVLRTKLMSTTRTVQREEIPSLVKSVQSKLSKNYVRGAKKILADAINKSAFQSSIASGFIGVCKSMGSKCVRDGKGGFKYIRDITSKIILHRDCHFCNQRKGVYCKAALGEVSEYSRPLIWDYFALVLTNACELGNWVFQKAEVPKIVTHLNNPNHFWPIKPSTHSELEDKVGINHILYSIRRNFPTLFDEHISPFLSDLNMLRLSWVQRIKFLDLCVAIDITSECLGIVSHIIKHRREELYIVKQNELAMSHSRESHPLERGFNLEPEEVCTNFLIQILFESMLVPVIMSTSQFKKYFWFGELELLPNNAQHDLKQLTQFICDCKKNNTSRTMNLDDLDVGFVSSKLILSCVNLNISVFINELDWVNRDNYENIEQLILASPSEVIPIELNLTFSHKRVSHKFRYERSTNYILKLRFLIERESLLDSLDSDGYLLLNPHSVEYYVSQSSGNHISLDGVSLLVLNPLINGKDVLDFNDLLEGQDIHFKSRSTVFQKVRIDLKNRFKDLKNKFSYKLIGPDVGMQPLILEGGLIKEGNRVVSRLEVNLDSKVVIIALEALEPEKRPRFIANLFQYLSSAQSHNKGISMNEQDLRLMIENFPEVFEHMLHDAKDWLNCGHFSIIRSKTLGSVMIADETGPFKIKGIRCRKLFEDNESVEIE

SEQ ID NO: 3 - protein, 561 aa, unidentified
  source 1..561: note = Pichinde virus
MSDNIPSFRWVQSLRRGLSNWTHPVKADVLSDTRALLSALDFHKVAQVQRMMRKDKRTDSDLTKLRDMNKEVDALMNMRSIQRDNVLKVGGLAKEELMELASDLDKLRKKVTRTESLSQPGVYGGNLTNTQLEQRAEILRSMGFANARPTGNRDGVVKIWDIKDNTLLINQFGSMPALTIACMTEQGGEQLNDVVQALSALGLLYTVKFPNMTDLEKLTQQHSALKIISNEPSAINISGYNLSLSAAVKAAACMIDGGNMLETIQVKPSMFSTLIKSLLQIKNREGMFVSTTPGQRNPYENLLYKICLSGDGWPYIGSRSQVQGRAWDNTTVDLDSKPSAIQPPVRNGGSPDLKQIPKEKEDTVVSSIQMLDSKATTWIDIEGTPNDPVEMAIYQPDTGNYIHCYRFPHDEKSFKEQSKYSHGLLLKDLADAQPGLISSIIRHLPQNMVFTAQGSDDIISLFEMHGRRDLKVLDVKLSAEQARTFEDEIWERYNLLCTKHKGLVIKKKKKGAAQTTANPHCALLDTIMFDATVTGWVRDQKPMRCLPIDTLYRNNTDLINL

SEQ ID NO: 4 - protein, 508 aa, unidentified
  source 1..508: note = Pichinde virus
MGQVVTLIQSIPEVLQEVFNVALIIVSTLCIIKGFVNLMRCGLFQLITFLILAGRSCDGMMIDRRHNLTHVEFNLTRMFDNLPQSCSKNNTHHYYKGPSNTTWGIELTLTNTSIANETTGNFSNIRSLAYGNISNCDKTEEAGHTLKWLLNELHFNVLHVTRHVGARCKTVEGAGVLIQYNLTVGDRGGEVGRHLIASLAQIIGDPKIAWVGKCFNNCSGGSCRLTNCEGGTHYNFLIIQNTTWENHCTYTPMATIRMALQKTAYSSVSRKLLGFFTWDLSDSTGQHVPGGYCLEQWAIVWAGIKCFDNTVMAKCNKDHNEEFCDTMRLFDFNQNAIKTLQLNVENSLNLFKKTINGLISDSLVIRNSLKQLAKIPYCNYTKFWYINDTITGRHSLPQCWLVHNGSYLNETHFKNDWLWESQNLYNEMLIKEYEERQGKTPLALTDICFWSLVFYTITVFLHLVGIPTHRHIIGDGCPKPHRITRNSLCSCGYYKIPKKPYKWVRLGK

SEQ ID NO: 5 - protein, 21 aa, synthetic construct
GSGEGRGSLLTCGDVEENPGP

SEQ ID NO: 6 - protein, 22 aa, synthetic construct
GSGATNFSLLKQAGDVEENPGP

SEQ ID NO: 7 - protein, 23 aa, synthetic construct
GSGQCTNYALLKLAGDVESNPGP

SEQ ID NO: 8 - protein, 25 aa, synthetic construct
GSGVKQTLNFDLLKLAGDVESNPGP

SEQ ID NO: 9 - protein, 566 aa, unidentified
  source 1..566: note = swine influenza virus
MKTIIALSYILCLVFAQKLPGNDNSTATLCLGHHAVPNGTLVKTITNDQIEVTNATELVQSSSTGRICDSPHRILDGKNCTLIDALLGDPHCDGFQNKEWDLFIERSKAYSNCYPYDVPDYSSLRSLVASSGTLEFTNEDFNWTGVAQDGGSYSCKRGSVKSFFSRLNWLHKLEYKYPALNVTMPNNDKFDKLYIWGVHHPSTDSEQTSLYVQAIGRVTVSTKSSQQTVIPNIGSRPWVRGISSRISIYWTIVKPGDILLISSTGNLIAPRGYFKIRNGKSSIMRSDAPIDNCYSECITPNGSIPNDKPFQNVNRITYGACPKYVKQKTLKLATGMRNVPEKQTRGIFGAIAGFIENGWEGMVDGWYGFRHQNSEGTGQAADLKSTQAAIDQVNGKLNRLIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 10 - protein, 565 aa, unidentified
  source 1..565: note = swine influenza virus
MKVKLLILLCTFTAAYADTICIGYHANNSTDTVDTVLEKNVTVTHSVNLLEDSHNGKLCLLKGMAPLQLGNCSVAGWILGNPECESLISKESWSYIVEAPNSDNGACYPGQFADYEELREQLSSVSSFERFEIFPRESSWPNHTVTGVSASCSHNGKRSFYRNLIWLTVKNGLYPNLSKSYENDKGKEVLILWGVHHPSNIGDQRTLYHTENAYVSVMSSHYSRRFTPEITKRPKVRDQEGRINYYWTLLEPGDTIIFETNGNLIAPWYAFALSRGLGSGIITSKAPMDKCDAKCQTPKGAINSNLPFQNVHPVTIGECPKYVRSTKLRMVTGLRNIPSIQSRGLFGAIAGFIEGGWTGMVDGWYGYHHQNEQGSGYAADQESTQNAINGITNKVNSVIEKMNTQFTAVGKEFNKLERRMENLNKKVDDGFLDIWTYNAELLVLLENERTLDFHDSNVKSLYEKVKSQLKNNAKEIGNGCFEFYHKCNNECMESVRNGTYDYPKYSEESKLNREKIDGVKLDSMGVYRILAIYSTVASSLVLLVSLGAISFWMCSNGSLQCRICI

SEQ ID NO: 11 - protein, 566 aa, unidentified
  source 1..566: note = swine influenza virus
MEAILVVLLYTFAAANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQGLYQNADAYVFVGSSRYSKKFKPEIAIRPKVRDQEGRMNYYWTLVEPGDKITFEATGNLVVPRYAFAMERNAGSGIIISDAPVHDCNTTCQTPNGAINTSLPFHNIHPITIGKCPKYVKSTKLRLATGLRNVPSIQSRGLFGAIAGFIEGGWTGMVDGWYGYHHQNEQGSGYAADLKSTQNAIDEVTNKVNSVIEKMNTQFTAVGKEFNHLETRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTKIYQILAIYSTVASSLVLVVSLGAISFWMCSNGSLQCRICI

SEQ ID NO: 12-14 - skipped (3 entries)

SEQ ID NO: 15 - RNA, 84 nt, synthetic construct
  source 1..84: mol_type = other RNA
cgcaccggggatcctaggcatctttgggtcacgcttcaaatttgtccaatttgaacccagctcaagtcctggtcaaaacttggg

SEQ ID NO: 16 - RNA, 30 nt, synthetic construct
  source 1..30: mol_type = other RNA
cgcaccgaggatcctaggcatttcttgatc

SEQ ID NO: 17 - RNA, 51 nt, synthetic construct
  source 1..51: mol_type = other RNA
cgcaccggggatcctaggcataccttggacgcgcatattacttgatcaaag

SEQ ID NO: 18 - RNA, 83 nt, synthetic construct
  source 1..83: mol_type = other RNA
cgcacagtggatcctaggcgattctagatcacgctgtacgttcacttcttcactgactcggaggaagtgcaaacaaccccaaa

SEQ ID NO: 19 - RNA, 71 nt, synthetic construct
  source 1..71: mol_type = other RNA
accagggcccctgggcgcacccccctccgggggtgcgcccgggggcccccggccccatggggccggttgtt

SEQ ID NO: 20 - RNA, 75 nt, synthetic construct
  source 1..75: mol_type = other RNA
gccctagcctcgacatgggcctcgacgtcactccccaataggggagtgacgtcgaggcctctgaggacttgagct

SEQ ID NO: 21-24 - skipped (4 entries)

SEQ ID NO: 25 - RNA, 87 nt, synthetic construct
  source 1..87: mol_type = other RNA
agctctcccttagccatccgagtggacgacgtcctccttcggatgcccaggtcggaccgcgaggaggtggagatgccatgccgaccc

(19) United States
(12) Patent Application Publication
(10) Pub. No.: US 2025/0082743 A1
(43) Pub. Date: Mar. 13, 2025

(54) VIRAL VECTORED VACCINES FOR SWINE INFLUENZA VIRUS AND METHODS OF USE

(71) Applicants: REGENTS OF THE UNIVERSITY OF MINNESOTA, Minneapolis, MN (US); BOARD OF REGENTS OF THE UNIVERSITY OF NEBRASKA, Lincoln, NE (US)

(72) Inventors: Hiep Lai Xuan Vu, Lincoln, NE (US); Hinh Ly, Woodbury, MN (US); Yuying Liang, Woodbury, MN (US)

(21) Appl. No.: 18/813,246

(22) Filed: Aug. 23, 2024

Related U.S. Application Data
(60) Provisional application No. 63/534,430, filed on Aug. 24, 2023.

(51) Int. Cl. A61K 39/145; A61K 39/00; A61P 37/04; C12N 15/86

(52) U.S. Cl. CPC A61K 39/145; A61P 37/04; C12N 15/86; A61K 2039/5256; A61K 2039/552; A61K 2039/575; C12N 2760/10043; C12N 2760/10051; C12N 2760/16122; C12N 2760/16134

(57) ABSTRACT

Provided herein are genetically engineered Pichinde viruses that include three ambisense genomic segments. Two of the genomic segments include an additional coding region that may encode one or more swine influenza virus (SIV) proteins, such as a SIV hemagglutinin (HA) protein. SIV HA proteins can be from different subtypes of SIV. Also provided herein is a reverse genetics system for making genetically engineered Pichinde virus, and a collection of vectors that can be used to produce genetically engineered Pichinde virus. Further provided are methods for using a reverse genetics system, and methods for treating a SIV infection in a subject.

### CROSS REFERENCE TO RELATED APPLICATIONS

This application claims the benefit of U.S. Provisional Application No. 63/534,430, filed Aug. 24, 2023, the disclosure of which is incorporated by reference herein in its entirety.

### GOVERNMENT FUNDING

This invention was made with government support under 1020750, and 2020-67015-31414 awarded by the National Institute of Food and Agriculture. The government has certain rights in the invention.”

### SEQUENCE LISTING

This application contains a Sequence Listing electronically submitted via Patent Center to the United States Patent and Trademark Office as an XML file entitled “0110.000712US01.xml” having a size of 24,727 bytes and created on Nov. 25, 2024. The information contained in the Sequence Listing is incorporated by reference herein.

### SUMMARY OF THE APPLICATION

Influenza A virus of swine (IAV-S, also referred to herein as SIV) is one of the most important respiratory pathogens of swine. The virus is widespread worldwide, causing significant economic loss to swine producers. Clinically, pigs infected with IAV-S often display signs of an acute respiratory disease that rapidly resolves after 7 or 10 days of infection. However, when associated with other pathogens of the porcine respiratory disease complex, IAV-S infection can often lead to severe pneumonia. There are three known major subtypes of IAV-S affecting pigs: H1N1, H1N2, and H3N2. Other less prevalent subtypes include H2N1, H3N1, and H2N3. Based on phylogenetic analysis of the IAV-S hemagglutinin (HA) sequences, the H1 and H3 subtypes can be further classified into multiple clades or lineages that are genetically distant from one another.

New strains of influenza virus frequently emerge in swine herds. Genetic diversity of IAV-S represents a major hurdle for developing an efficacious vaccine.

The inventors have determined that a vector system based on the Pichinde virus can be used in pigs to result in protective immunity to SIV. Described herein are results showing that pigs vaccinated with a recombinant Pichinde virus (rPICV) vectored vaccine expressing the hemagglutinin (HA) gene of an H3N2 strain of influenza A virus of swine generated virus-neutralizing antibodies and were protected against infection with the matching H3N2 strain. Also described herein is a trivalent rPICV-vectored vaccine that successfully elicits antibody responses against the three targeted IAV-S strains and provides protection in pigs.

Terms used herein will be understood to take on their ordinary meaning in the relevant art unless specified otherwise. Several terms used herein and their meanings are set forth below.

As used herein, “genetically modified” and “genetically engineered” refers to a Pichinde virus which has been modified and is not found in any natural setting. For example, a genetically modified Pichinde virus is one into which has been introduced an exogenous polynucleotide, such as a restriction endonuclease site. Another example of a genetically modified Pichinde virus is one which has been modified to include three genomic segments.

A “coding region” is a nucleotide sequence that encodes an RNA molecule. The boundaries of a coding region are generally determined by a transcription initiation site at its 5′ end and a transcription terminator at its 3′ end. A coding region typically includes at least one nucleotide sequence that encodes a protein. A nucleotide sequence encoding a protein, also referred to as an open reading frame (ORF) has boundaries that are generally determined by a translation start codon at its 5′ end and a translation stop codon at its 3′ end. A coding region can encode an RNA molecule that includes one or more open reading frames. An RNA molecule that includes one open reading frame is referred to as a “monocistronic message.” An RNA molecule that includes two or more open reading frames is referred to as a “polycistronic message.”

As used herein, the term “protein” refers broadly to a polymer of two or more amino acids joined together by peptide bonds. The term “protein” also includes molecules which contain more than one protein joined by disulfide bonds, ionic bonds, or hydrophobic interactions, or complexes of proteins that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long-term culture in tissue culture medium). “In vivo” refers to cells that are within the body of a subject.

While polynucleotide sequences described herein are listed as DNA or RNA sequences, it is understood that the complements, reverse sequences, and reverse complements of the DNA and RNA sequences can be easily determined by the skilled person. It is also understood that the sequences disclosed herein as DNA sequences can be converted from a DNA sequence to an RNA sequence by replacing each thymidine nucleotide with a uridine nucleotide. Likewise, the sequences disclosed herein as RNA sequences can be converted from a RNA sequence to an DNA sequence by replacing each uridine nucleotide with a thymidine nucleotide.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Conditions that are “suitable” for an event to occur, such as generating full-length genomic RNA molecules of a genomic segment, or “suitable” conditions are conditions that do not prevent such events from occurring. Thus, these conditions permit, enhance, facilitate, and/or are conducive to the event.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIGS. 1A-B show animal study design. (FIG. 1A) Table indicating different treatment groups that were included in the study along with dosage specification for vaccine and challenge virus. (FIG. 1B) schematic view of study schedule representing duration at which vaccination, challenge, and sample collections were carried out. Image was made using the Biorender tool.

FIGS. 2A-D show humoral antibody responses post vaccination. (FIG. 2A) Anti-GFP antibodies in plasma samples from the T01 (PBS) and T02 (rPICV-GFP) groups. (FIG. 2B) Anti-H3 antibodies in plasma samples from all treatment groups. An arbitrary cutoff equivalent to mean plus 5 standard deviations of OD values of samples from the T01 and T02 groups was calculated. Samples with OD values greater than the cutoff were considered to be positive. The end-point antibody titers were expressed as the log 2 of the reciprocal of the highest plasma dilution that tested positive by the H3-specific ELISA. (FIG. 2C) Virus neutralization (VN) antibody titers against H3N2 TX98. (FIG. 2D) Hemagglutination inhibition antibody titers against H3N2 TX98. In (FIG. 2C, FIG. 2D), the horizontal dotted lines at 5.32 log 2 (1:40 dilution) indicate the background antibody titers found in all pigs prior to vaccination. T01-PBS; T02-rPICV-GFP; T03-rPICV-H3; T04-H3-protein.

FIGS. 3A-D show IAV-S shedding post challenge. (FIG. 3A) IAV-S NP RNA in nasal swab samples as quantified by RT-PCR. Data are expressed as log 10 RNA copy per microliter of RNA sample loaded to the PCR reaction. (FIG. 3B) Area under each curve of the nasal viral loads in pigs in the course of 5 days post challenge infection with IAV-S. (FIG. 3C) IAV-S NP RNA in BALF collected during necropsy at day 5 post challenge. (FIG. 3D) Infectious IAV-S in BALF samples measured by virus titration in MDCK cells. T01-PBS; T02-rPICV-GFP; T03-rPICV-H3; T04-H3-protein. ns-no significance, ** p less than or equal to 0.01, *** p less than or equal to 0.001, **** p less than or equal to 0.0001.

FIGS. 4A-C show Lung macroscopic and microscopic lesion. (FIG. 4A) Percentage consolidation calculated on a weighted average of each lung lobe. (FIG. 4B) Composite microscopic lesion scores based on parameters described previously (Gauger et al. Virology 2014, 471-473, 93-104). Sections from three different lung lobes (apical, middle, and caudal) of each pig were scored, and the average composite score of each pig was used for statistical analysis. (FIG. 4C) Composite ISH scores based on parameters described in (Gauger et al. Virology 2014, 471-473, 93-104). T01-PBS; T02-rPICV-GFP; T03-rPICV-H3; T04-H3-protein. ns-no significance; * p less than or equal to 0.05; ** p less than or equal to 0.01; *** p less than or equal to 0.001.

FIG. 5 shows antibodies against IAV-S NP protein measured by a blocking ELISA. Data are expressed as the sample to negative (S/N) ratio. The horizontal dotted line at S/N of 0.6 is the assay cutoff. Test samples with S/N greater than 0.6 are considered to be negative. T01-PBS; T02-rPICV-GFP; T03-rPICV-H3; T04-H3-protein.

FIGS. 6A-G show antibody responses after vaccination. (FIG. 6A) Antibodies against IAV-S NP. Data are expressed as the sample-to-negative (S/N) ratio. A dotted line at S/N of 0.6 represents the cut-off limit for the assay. Samples with S/N value above the cutoff line are considered negative for the NP antibodies. NV/NC, neither vaccinated nor challenged. PBS, mock vaccination. WIV, whole-inactivated virus vaccine. TPV, trivalent PICV-vectored vaccine. (FIG. 6B-D) Virus-neutralizing antibody titers against the H3N2-TX98, H1N2-1a, and H1N1-pdm09 strains, respectively. (FIG. 6E-G) Hemagglutination inhibition (HI) antibody titers measured against the H3N2-TX98, H1N2-1a, and H1N1-pdm09 strains, respectively. For the VN and HI, the dotted lines at the dilution of 1:10 indicate the assay's detection limit. Samples with undetectable activity at this dilution were considered negative and were assigned a value of 5 for graphical and statistical purposes. Results are displayed in the order of NV/NC, PBS, WIV, TPV at each time point. ns: no significant, * p≤0.05; ** p≤0.01; *** p≤0.001; **** p≤0.0001.

FIGS. 7A-C show results relating to viral genomic RNA shedding in pigs post-challenge with H1N1-pdm09 virus. (FIG. 7A) IAV-S genomic RNA copies in nasal swabs. (FIG. 7B) The area under the curve of viral loads in the nasal swabs. (FIG. 7C) IAV-S genomic RNA copies in bronchoalveolar lavage fluid (BALF) collected on day 5 p.c. The dotted horizontal lines at y=0.8 log 10 represent the assay detection limit. ns: non-significant, * p≤0.05; p≤0.01; *** p≤0.001.

FIGS. 8A-C show results of pathological assessment of pig lungs after challenge infection with the H1N1-pdm09 virus. (FIG. 8A) Percentage of lung consolidation calculated based on the weighted proportions of each lobe to the total lung volume. (FIG. 8B) Composite score of microscopic lung lesion. (FIG. 8C) Composite scores of the lung section stained with ISH. ns: non-significant, * p≤0.05; ** p≤0.01; *** p≤0.001.

FIGS. 9A, 9B, and 9C show certain amino acid sequences discussed in the present disclosure.

### DETAILED DESCRIPTION

Provided herein is a reverse genetics system for producing genetically modified Pichinde virus that expresses proteins of swine influenza virus (SIV). The genetically modified Pichinde virus-based reverse genetics system described herein has multiple advantages over other arenavirus systems for expression of SIV proteins and use in immunizing subjects, including genetic stability of the modified Pichinde virus through serial passages in cell cultures.

The reverse genetics system for this modified Pichinde virus includes three genomic segments. The first genomic segment includes two coding regions, one that encodes a Z protein and a second that encodes a RNA-dependent RNA polymerase (L RdRp). The second genomic segment includes a coding region that encodes a nucleoprotein (NP), and one or more additional coding regions that encode one or more SIV proteins. The third genomic segment includes a coding region that encodes a glycoprotein, and one or more additional coding regions that encode one or more SIV proteins. The additional coding regions can be expressed as a monocistronic mRNA molecule that encodes either one protein or two or more proteins, or as a polycistronic mRNA. Monocistronic and polycistronic mRNAs are described herein.

The Z protein, L RdRp, NP protein, and glycoprotein are those encoded by a Pichinde virus. The Z protein is a small RING-domain containing matrix protein that mediates virus budding and regulates viral RNA synthesis. One example of a Z protein from a Pichinde virus is the sequence available at Genbank accession number ABU39910.1 (SEQ ID NO:1). The L RdRp protein is a RNA-dependent RNA polymerase that is required for viral DNA synthesis. One example of a L RdRp protein from a Pichinde virus is the sequence available at Genbank accession number ABU39911.1 (SEQ ID NO:2). The NP protein encapsidates viral genomic RNAs, is required for viral RNA synthesis, and suppresses host innate immune responses. One example of a NP protein from a Pichinde virus is the sequence available at Genbank accession number ABU39909.1 (SEQ ID NO:3). The glycoprotein is post-translationally processed into a stable signal peptide (SSP), the receptor-binding G1 protein, and the transmembrane G2 protein. One example of a glycoprotein from a Pichinde virus is the sequence available at Genbank accession number ABU39908.1 (SEQ ID NO:4).

Other examples of Z proteins, L RdRp proteins, NP proteins, and glycoprotein include proteins having structural similarity with a protein that is encoded by a Pichinde virus, for instance, SEQ ID NO:1, 2, 3, and/or 4. Structural similarity of two proteins can be determined by aligning the residues of the two proteins (for example, a candidate protein and a reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein, such as SEQ ID NO:1, 2, 3, or 4. A candidate protein is the protein being compared to the reference protein. A candidate protein may be isolated, for example, from a cell of an animal, such as a mouse, or can be produced using recombinant techniques, or chemically or enzymatically synthesized. A candidate protein may be inferred from a nucleotide sequence present in the genome of a Pichinde virus.

Unless modified as otherwise described herein, a pair-wise comparison analysis of amino acid sequences can be carried out using the Blastp program of the blastp suite-2sequences search algorithm, as described by Tatusova et al., (FEMS Microbiol Lett, 174, 247-250 (1999)), and available on the National Center for Biotechnology Information (NCBI) website. The default values for all blastp suite-2sequences search parameters may be used, including general parameters: expect threshold=10, word size=3, short queries=on; scoring parameters: matrix=BLOSUM62, gap costs=existence: 11 extension: 1, compositional adjustments=conditional compositional score matrix adjustment. Alternatively, proteins may be compared using the BESTFIT algorithm in the GCG package (version 10.2, Madison WI).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free-OH is maintained; and Gln for Asn to maintain a free —NH2.

The skilled person will recognize that the Z protein depicted at SEQ ID NO:1 can be compared to Z proteins from other arenaviruses, including Lassa virus (073557.4), LCMV Armstrong (AAX49343.1), and Junin virus (NP_899216.1) using readily available algorithms such as ClustalW to identify conserved regions of Z proteins. ClustalW is a multiple sequence alignment program for nucleic acids or proteins that produces biologically meaningful multiple sequence alignments of different sequences (Larkin et al., 2007, ClustalW and ClustalX version 2, Bioinformatics, 23 (21): 2947-2948). Using this information, the skilled person can readily predict with a reasonable expectation that certain conservative substitutions to an Z protein such as SEQ ID NO:1 will not decrease activity of the protein.

The skilled person will recognize that the L RdRp protein depicted at SEQ ID NO:2 can be compared to L RdRp proteins from other arenaviruses, including Lassa virus (AAT49002.1), LCMV Armstrong (AAX49344.1), and Junin virus (NP_899217.1) using readily available algorithms such as ClustalW to identify conserved regions of L RdRp proteins. Using this information, the skilled person can readily predict with a reasonable expectation that certain conservative substitutions to an L RdRp protein such as SEQ ID NO:2 will not decrease activity of the protein.

The skilled person will recognize that the NP protein depicted at SEQ ID NO:3 can be compared to NP proteins from other arenaviruses, including Lassa virus (P13699.1), LCMV Armstrong (AAX49342.1), and Junin virus (NP_899219.1) using readily available algorithms such as ClustalW to identify conserved regions of NP proteins. Using this information, the skilled person can readily predict with a reasonable expectation that certain conservative substitutions to a NP protein such as SEQ ID NO:3 will not decrease activity of the protein.

The skilled person will recognize that the glycoprotein depicted at SEQ ID NO:4 can be compared to glycoproteins from other arenaviruses, including Lassa virus (P08669), LCMV Armstrong (AAX49341.1), and Junin virus (NP_899218.1) using readily available algorithms such as ClustalW to identify conserved regions of glycoproteins. Using this information, the skilled person can readily predict with a reasonable expectation that certain conservative substitutions to a glycoprotein such as SEQ ID NO:4 will not decrease activity of the protein.

Thus, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence. Alternatively, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence. Unless noted otherwise, “Z protein,” “L RdRp protein,” “NP protein,” and “glycoprotein” refer to a protein having at least 80% amino acid identity to SEQ ID NO:1, SEQ ID NO:2, SEQ ID NO:3, and SEQ ID NO: 4, respectively.

A Z protein, L RdRp protein, an NP protein, or a glycoprotein having structural similarity the amino acid sequence of SEQ ID NO: 1, 2, 3, or 4, respectively, has biological activity. As used herein, “biological activity” refers to the activity of Z protein, L RdRp protein, an NP protein, or a glycoprotein in producing an infectious virus particle. The biological role each of these proteins play in the biogenesis of an infectious virus particle is known, as are assays for measuring biological activity of each protein.

In one embodiment, the NP protein may include one or more mutations. A mutation in the NP protein may result in a NP protein that continues to function in the production of infectious viral particles but has a decreased ability to suppress the production of certain cytokines by a cell infected with a Pichinde virus. A Pichinde virus that has decreased ability to suppress cytokine production is expected to be useful in enhancing an immunological response to a protein encoded by the virus. Examples of mutations include the aspartic acid at residue 380, the glutamic acid at residue 382, the aspartic acid at residue 457, the aspartic acid at residue 525, and the histidine at residue 520. A person of ordinary skill in the art recognizes that the precise location of these mutations can vary between different NP proteins depending upon the presence of small insertions or deletions in the NP protein, thus the precise location of a mutation is approximate, and can vary by 1, 2, 3, 4, 5, or more amino acids.

In one embodiment, the mutation in the NP protein may be the replacement of the aspartic acid, glutamic acid, or histidine at residues 380, 382, 457, 525, and/or 520 with any other amino acid. In one embodiment, the mutation may be the conservative substitution of the aspartic acid, glutamic acid, or histidine at residues 380, 382, 457, 525, and/or 520. In one embodiment, the mutation may be the replacement of the aspartic acid, glutamic acid, or histidine at residues 380, 382, 457, 525, and/or 520 with a glycine or an alanine. In one embodiment, the NP protein may include a mutation at one, two, three, or four of the residues 380, 382, 457, 525, or 520, and in one embodiment the NP protein may include a mutation at all five residues.

In one embodiment, the glycoprotein may include one or more mutations. A mutation in the glycoprotein may result in a glycoprotein that impairs virus spreading in vivo. Examples of mutations include the asparagine at residue 20, and/or the asparagine at residue 404. A person of ordinary skill in the art recognizes that the precise location of these mutations can vary between different glycoproteins depending upon the presence of small insertions or deletions in the glycoprotein, thus the precise location of a mutation is approximate, and can vary by 1, 2, 3, 4, or 5 amino acids.

In one embodiment, the mutation in the glycoprotein may be the replacement of the asparagine residue 20 and/or 404 with any other amino acid. In one embodiment, the mutation may be the conservative substitution of the asparagine residue 20 and/or 404. In one embodiment, the mutation may be the replacement of the asparagine residue 20 and/or 404 with a glycine or an alanine.

In some embodiments, the second genomic segment and third genomic segment each independently include one or more additional coding regions that include an open reading frame encoding one or more SIV proteins. The one or more additional coding regions present on a genomic segment can be expressed as a monocistronic mRNA or a polycistronic mRNA. A monocistronic mRNA includes one open reading frame. As described herein, the open reading frame can encode one or more proteins. A polycistronic mRNA is one that includes two or more open reading frames, where each open reading frame encodes a protein. A genomic segment can include one or more coding regions that are expressed as a monocistronic mRNA molecule, one or more coding regions that are expressed as a polycistronic mRNA molecule, or a combination of coding regions where one or more are expressed as a monocistronic mRNA and one or more are expressed as a polycistronic mRNA. In one embodiment, a genomic segment includes an open reading frame encoding a Z protein, an open reading frame encoding a L RdRp protein, an open reading frame encoding a NP protein, an open reading frame encoding a glycoprotein, and at least one coding region that is expressed as a monocistronic mRNA molecule that encodes either one protein or two or more proteins, or any combination thereof.

In one embodiment, a monocistronic message includes an open reading frame that encodes a protein. In another embodiment, a monocistronic message includes separate regions that encode proteins, but each separate region does not include a translation start codon at its 5′ end and a translation stop codon at its 3′ end. Instead, the nucleotides between the separate regions that encode proteins encode a self-cleaving peptide. An example of a class of self-cleaving peptide is the 2A peptide. 2A peptides are amino acid sequences that can result in a molecular event after translation that has the effect of cleaving the amino acid sequence. Four examples of 2A peptides are shown in Table 1. The cleavage is trigged by breaking a peptide bond between the Proline (P) and Glycine (G) in the C-terminal end of the 2A peptide. The molecular mechanism of 2A-peptide-mediated cleavage is unknown but is believed to involve ribosomal “skipping” of glycyl-prolyl peptide bond formation rather than true proteolytic cleavage (Wang et al., 2015, Scientific Reports. 5 (1), doi: 10.1038/srep16273, ISSN 2045-2322; Ryan et al., 2001, Journal of General Virology. 82 (5): 1013-1025; and Sharma et al., 2012, Nucleic Acids Research. 40 (7): 3143-3151).

TABLE 1
2A peptides

| Peptide | Amino acid sequence¹ |
|---|---|
| T2A | (GSG)EGRGSLLTCGDVEENPGP (SEQ ID NO: 5) |
| P2A | (GSG)ATNFSLLKQAGDVEENPGP (SEQ ID NO: 6) |
| E2A | (GSG)QCTNYALLKLAGDVESNPGP (SEQ ID NO: 7) |
| F2A | (GSG)VKQTLNFDLLKLAGDVESNPGP |
| | (SEQ ID NO: 8) |

¹Addition of the GSG residues at the 5′ end of the peptide is optional and can improve cleavage efficiency.

In one embodiment, a polycistronic message includes separate open reading frames, e.g., one or more open reading frames within a polycistronic message each having a translation start codon at its 5′ end and a translation stop codon at its 3′ end. Each coding region is separately translated beginning at the translation start codon and ending at the translation stop codon. An intercistronic region can be located between two of the coding regions. An intercistronic region is at least one nucleotide that is not translated into a protein.

In one embodiment, the number of SIV proteins encoded by a genomic segment can be 1, 2, 3, 4, 5, 6, 7, 8, 9, 10, or more. The proteins can be present as open reading frames that are expressed as a monocistronic mRNA or a polycistronic mRNA. A factor that influences the number of coding regions is the total length of the genomic segments. As described herein, in some embodiments it is useful to rescue virus and produce an infectious virus particle. In one embodiment, when the second and/or third genomic segments include one or more additional coding regions encoding one or more proteins, the maximum size in nucleotides of the added coding region(s) to a genomic segment is no greater than 2 kilobases (kb), no greater than 1.8 kb, or no greater than 1.6 kb.

A SIV protein useful herein results in a humoral immune response, a cell-mediated immune response, or a combination thereof when expressed in a subject. In one embodiment, the protein is at least 6 amino acids in length. The nucleotide sequence encoding a SIV protein can be modified to reflect the codon usage bias of a cell in which the Pichinde virus and SIV protein(s) will be expressed. The usage bias of nearly all cells in which a Pichinde virus would be expressed is known to the skilled person. Any SIV protein that results in a humoral immune response, a cell-mediated immune response, or a combination thereof when expressed in a subject can be used. Specific non-limiting examples of SIV proteins are described herein.

Examples of SIV proteins that can be expressed include a hemagglutinin (HA). Examples of hemagglutinins include those from different SIV subtypes. An example of a SIV hemagglutinin from a H3N2 subtype is SEQ ID NO:9 (available at Genbank accession number AEK70342.1). An example of a SIV hemagglutinin from a H1N2 subtype is SEQ ID NO: 10 (available at Genbank accession number AGZ62252.1). An example of a SIV hemagglutinin from a H1N1 subtype is SEQ ID NO:11 (available at Genbank accession number).

The person of ordinary skill will recognize that other SIV hemagglutinin proteins are readily available and can be easily identified. For instance, a SIV can be isolated from a pig infected with the virus and the nucleotide sequence of the virus determined using routine methods. The nucleotide sequence can be evaluated and the location of the sequence encoding the desired protein easily identified. That sequence can be inserted into to viral system for expression as described herein.

Other examples of SIV hemagglutinins include those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to SEQ ID NO:9, SEQ ID NO: 10, or SEQ ID NO:11.

Pichinde virus is an arenavirus, and one characteristic of an arenavirus is an ambisense genome. As used herein, “ambisense” refers to a genomic segment having both positive sense and negative sense portions and coding strategies. For example, the first genomic segment of a Pichinde virus described herein is ambisense, encoding a Z protein in the positive sense and encoding a L RdRp protein in the negative sense. Thus, one of the two coding regions of the first genomic segment is in a positive-sense orientation and the other is in a negative-sense orientation. When the second and/or the third genomic segment includes a second coding region encoding a protein, e.g., a SIV protein, the coding region encoding the protein is in a negative-sense orientation compared to the NP protein of the second genomic segment and to the glycoprotein of the third genomic segment.

Each genomic segment also includes nucleotides encoding a 5′ untranslated region (UTR) and a 3′ UTR. These UTRs are located at the ends of each genomic segment. Nucleotides useful as 5′ UTRs and 3′ UTRs include those present in Pichinde virus and are readily available to the skilled person (see, for instance, Buchmeier et al., 2007, Arenaviridae: the viruses and their replication. In: Knipe and Howley (eds), Fields Virology. 5th ed. Philadelphia, PA: Lippincott Williams & Wilkins. pp. 1791-1827). In one embodiment, a genomic segment that encodes a Z protein and an L RdRp protein includes a 5′ UTR sequence that is 5′ CGCACCGGGGAUCCUAGGCAUCUUUGGGUCACGCUUCAAAUUUGUCCAAUU UGAACCCAGCUCAAGUCCUGGUCAAAACUUGGG (SEQ ID NO:15) and a 3′ UTR sequence that is CGCACCGAGGAUCCUAGGCAUUUCUUGAUC (SEQ ID NO:16). In one embodiment, a genomic segment that encodes a NP protein or a glycoprotein includes a 5′ UTR sequence that is 5′ CGCACCGGGGAUCCUAGGCAUACCUUGGACGCGCAUAUUACUUGAUCAAAG (SEQ ID NO:17) and a 3′ UTR sequence that is 5′ CGCACAGUGGAUCCUAGGCGAUUCUAGAUCACGCUGUACGUUCACUUCUUCA CUGACUCGGAGGAAGUGCAAACAACCCCAAA (SEQ ID NO:18). Alterations in these sequences are permitted, and the terminal 27-30 nucleotides are highly conserved between the genomic segments.

Each genomic segment also includes an intergenic region (IGR) located between the coding region encoding a Z protein and the coding region encoding a L RdRp protein, between the coding region encoding a nucleoprotein and the additional coding region(s), and between the coding region encoding a glycoprotein and the additional coding region(s). Nucleotides useful as an intergenic region are those present in Pichinde virus and are readily available to the skilled person. In one embodiment, an IGR sequence of a genomic segment that encodes a Z protein and an L RdRp protein includes 5′ ACCAGGGCCCCUGGGCGCACCCCCCUCCGGGGGUGCGCCCGGGGGCCCCCGG CCCCAUGGGGCCGGUUGUU (SEQ ID NO:19). In one embodiment, an IGR sequence of a genomic segment that encodes a NP protein or a glycoprotein includes 5′ GCCCUAGCCUCGACAUGGGCCUCGACGUCACUCCCCAAUAGGGGAGUGACGU CGAGGCCUCUGAGGACUUGAGCU (SEQ ID NO:20).

In one embodiment, a coding region can include nucleotides that encode a protein that is useful as a detectable marker, e.g., a molecule that is easily detected by various methods. Examples include fluorescent proteins (e.g., green, yellow, blue, or red fluorescent proteins), luciferase, chloramphenicol acetyl transferase, and other molecules (such as c-myc, flag, 6×his, HisGln (HQ) metal-binding peptide, and V5 epitope) detectable by their fluorescence, enzymatic activity or immunological properties.

In one embodiment, one genomic segment includes an open reading frame encoding a Z protein and an open reading frame encoding a L RdRp protein. In one embodiment, one genomic segment includes an open reading frame encoding a NP protein and one coding region that is expressed as a monocistronic mRNA molecule that encodes either one protein or two or more proteins. In one embodiment, one genomic segment includes an open reading frame encoding a glycoprotein and one coding region that is expressed as a monocistronic mRNA molecule that encodes either one protein or two or more proteins. In those embodiments where a monocistronic mRNA molecule encodes two or more proteins, a self-cleaving peptide can be present between the different proteins.

One or more of the genomic segments described herein can be present in a vector. For instance, all genomic segments can be present in one vector, two can be present in one vector, or each genomic segment can be present in different vectors. In one embodiment, the sequence of a genomic segment in the vector is antigenomic, and in one embodiment the sequence of a genomic segment in the vector is genomic. As used herein, “anti-genomic” refers to a genomic segment that encodes a protein in the orientation opposite to the viral genome. For example, Pichinde virus is a negative-sense RNA virus. However, each genomic segment is ambisense, encoding proteins in both the positive-sense and negative-sense orientations. “Anti-genomic” refers to the positive-sense orientation, while “genomic” refers to the negative-sense orientation.

A vector is a replicating polynucleotide, such as a plasmid, phage, or cosmid, to which another polynucleotide may be attached so as to bring about the replication of the attached polynucleotide. Construction of vectors containing a genomic segment, and construction of genomic segments including insertion of a polynucleotide encoding a protein, employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of an RNA encoded by the genomic segment, i.e., an expression vector. The term vector includes, but is not limited to, plasmid vectors, viral vectors, cosmid vectors, or artificial chromosome vectors. Typically, a vector is capable of replication in a prokaryotic cell and/or a eukaryotic cell. In one embodiment, the vector replicates in prokaryotic cells, and not in eukaryotic cells. In one embodiment, the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells.

An expression vector optionally includes regulatory sequences operably linked to the genomic segment. The term “operably linked” refers to a juxtaposition of components such that they are in a relationship permitting them to function in their intended manner. A regulatory sequence is “operably linked” to a genomic segment when it is joined in such a way that expression of the genomic segment is achieved under conditions compatible with the regulatory sequence. One regulatory sequence is a promoter, which acts as a regulatory signal that bind RNA polymerase to initiate transcription of the downstream (3′ direction) genomic segment. The promoter used can be a constitutive or an inducible promoter. The present disclosure is not limited by the use of any particular promoter, and a wide variety of promoters are known. In one embodiment, a T7 promoter is used. Another regulatory sequence is a transcription terminator located downstream of the genomic segment. Any transcription terminator that acts to stop transcription of the RNA polymerase that initiates transcription at the promoter may be used. In one embodiment, when the promoter is a T7 promoter, a T7 transcription terminator is also used. In one embodiment, a ribozyme is present to aid in processing an RNA molecule. A ribozyme may be present after the sequences encoding the genomic segment and before a transcription terminator. An example of a ribozyme is a hepatitis delta virus ribozyme. One example of a hepatitis delta virus ribozyme 5′ is AGCTCTCCCTTAGCCATCCGAGTGGACGACGTCCTCCTTCGGATGCCCAGGTCG GACCGCGAGGAGGTGGAGATGCCATGCCGACCC (SEQ ID NO:25).

Transcription of a genomic segment present in a vector results in an RNA molecule. When each of the three genomic segments is present in a cell the coding regions of the genomic segments are expressed and viral particles that contain one copy of each of the genomic segments are produced. The three genomic segments of the reverse genetics system described herein are based on Pichinde virus, an arenavirus with a segmented genome of two single-stranded ambisense RNAs. While the ability of the reverse genetics system to replicate and produce infectious virus typically requires the presence of the ambisense RNAs in a cell, the genomic segments described herein also include the complement thereof (i.e., complementary RNA), and the corresponding DNA sequences of the two RNA sequences.

A polynucleotide used to transform a host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, and neomycin.

Also provided are compositions including a viral particle described herein, or the three genomic segments described herein. Such compositions may include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition described herein may be referred to as a vaccine. The term “vaccine” as used herein refers to a composition that, upon administration to an animal, will increase the likelihood the recipient mounts an immune response to a SIV protein encoded by one of the genomic segments described herein.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational (e.g., intranasal), transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, phosphate buffered saline (PBS), and the like. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a viral particle described herein) in the required amount in an appropriate solvent with one or a combination of ingredients enumerated above, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds can be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

The data obtained from cell culture assays and animal studies can be used in formulating a range of dosage for use in an animal. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

The compositions can be administered once to result in an immune response, or one or more additional times as a booster to potentiate the immune response and increase the likelihood immunity to the proteins is long-lasting. The skilled artisan will appreciate that certain factors may influence the dosage and timing required to effectively treat a subject, including but not limited to the severity of the disease or disorder, previous treatments, the general health and/or age of the subject, and other diseases present.

Also provided herein are methods for using the genomic segments. In one embodiment, a method includes making an infectious viral particle. Such a method includes, but is not limited to, providing a cell, such as an ex vivo cell, that includes each of the three genomic segments described herein (a first genomic segment, a second genomic segment, and a third genomic segment) and incubating the cell under conditions suitable for generating full-length genomic RNA molecules of each genomic segment. The full-length genomic RNA of each genomic segment is antigenomic.

Production of full-length genomic RNA molecules of each genomic segment results in transcription and translation of each viral gene product and amplification of the viral genome to generate infectious progeny virus particles. As used herein, an “infectious virus particle” refers to a virus particle that can interact with a suitable eukaryotic cell, such as a mammalian cell (e.g., a murine cell or a porcine cell) or an avian cell, to result in the introduction of the three genomic segments into the cell, and the transcription of the three genomic segments in the cell. The method can also include introducing into the cell vectors that encode the three genomic segments. Infectious virus particles are released into a supernatant and may be isolated and amplified further by culturing on a eukaryotic cell, such as, but not limited to, ex vivo baby hamster kidney (BHK21) epithelial cells or African green monkey epithelial (VERO) cells. The method may include isolating a viral particle from a cell or a mixture of cells and cellular debris. The method may include inactivating virus particles using standard methods, such a hydrogen peroxide treatment. Also provided is a viral particle, infectious or inactivated, that contains three genomic segments described herein.

In one embodiment, a method includes expression of one or more SIV proteins in a cell. Such a method includes, but is not limited to, introducing into a cell the three genomic segments described herein. In one embodiment, the introducing is by introduction of a virus particle that is infectious or inactivated. The second and/or the third genomic segment may include one or more additional coding regions encoding one or more SIV proteins. More than one type of virus particle may be administered. For instance, two populations, three populations, or four or more populations of virus particles may be administered where each population encodes different SIV proteins. The use of two or more populations of virus particles can be advantageous when a herd of swine are infected by two or more different subtypes of SIV. Each population of virus particles can be directed to one of the different SIV subtypes. The cell is a suitable eukaryotic cell, such as a mammalian cell (e.g., a murine cell or a porcine cell) or an avian cell. In one embodiment, the avian cell is a chicken embryonic fibroblast. The cell may be ex vivo or in vivo. The three genomic segments may be introduced by contacting a cell with an infectious virus particle that contains the three genomic segments, or by introducing into the cell vectors that include the genomic segments. In one embodiment, the introducing includes administration of a composition that includes one or more populations of infectious virus particles that contain the three genomic segments, where each population encodes a different SIV protein. The method further includes incubating the cell under conditions suitable for expression of the coding regions present on the three genomic segments.

In one embodiment, a method includes immunizing an animal and/or treating a SIV infection in an animal. Such a method includes, but is not limited to, administering to an animal a viral particle that is infectious or inactivated, that contains the three genomic segments described herein. In one embodiment, one type of virus particle may be administered. The virus particle can include coding regions encoding one or two of the same SIV HA protein, such as a SIV HA protein from SIV subtype H1N1, H1N2, or H3N2. The HA protein can provide homologous protection (the recipient animal has increased immunity to SIV of the same subtype as the encoded and expressed HA protein). In some embodiments, the HA protein can provide heterologous protection (the recipient animal has increased immunity to SIV of a different subtype than the encoded and expressed HA protein). Heterologous protection can be evaluated using the hemagglutination inhibition assay (Example 4) or by immunizing an animal with a virus particle encoding a HA of a specific subtype and then challenging the animal with an SIV of a different subtype. Immunization using a viral particle encoding a HA of subtype H1N1, H1N2, or H3N2 may result in heterologous protection against SIV isolates within subtypes H2N1, H3N1, and/or H2N3.

In some embodiments, a method for immunizing an animal and/or treating a SIV infection in an animal can include administration of more than one type of virus particle. For instance, three populations of virus particles may be administered where each population encodes different SIV proteins, e.g., HA proteins from the same SIV subtype. In another example, the three populations of virus particles administered can encode SIV proteins that result in immunity for different SIV subtypes, e.g., the encoded HA proteins are from different SIV subtypes, such as any combination of H1N1, H1N2, and H3N2.

The animal receiving the administration is a member of the kingdom Animalia, and may be any animal in need of immunization, including a vertebrate, such as a mammal. The animal can be, for instance, a porcine animal, such as a domesticated pig a mouse, a guinea pig, or a rabbit. In one embodiment, the animal is a human. In one embodiment, the animal is an animal at risk of exposure to SIV. In another embodiment, the animal is an animal that has a SIV infection. The administration may be part of a routine standard of care in proactively preventing the likelihood of infection. The immune response may be a humoral response (e.g., the immune response includes production of antibody in response to an antigen), a cellular response (e.g., the activation of phagocytes, antigen-specific cytotoxic T-lymphocytes, and the release of cytokines in response to an antigen), or a combination thereof. In one embodiment, genetically modified Pichinde virus-based reverse genetics system described herein can be used to protect a human from swine flu.

As used herein, the term “infection” refers to the presence of and multiplication of SIV in the body of a subject. The infection can be clinically inapparent or result in signs associated with SIV. The infection can be at an early stage, or at a late stage. In another embodiment, a method includes treating one or more symptoms of SIV in an animal. In one embodiment, the method includes administering an effective amount of a composition described herein to an animal having or at risk of having a SIV infection. Optionally the method includes determining whether the amount of SIV in the animal decreases. In one embodiment, the method includes administering an effective amount of a composition described herein to an animal having at least one sign of SIV, and determining whether at least one sign of the condition is reduced.

Treatment of a sign associated with SIV can be prophylactic or, alternatively, can be initiated after the development of SIV. As used herein, the term “sign” refers to objective evidence in a subject of a condition caused by infection by disease. Signs associated with SIV and the evaluations of such signs is routine and known in the art. Treatment that is prophylactic, for instance, initiated before a subject manifests signs of SIV, is referred to herein as treatment of a subject that is “at risk” of developing SIV. An animal at risk of SIV is one that may be exposed to SIV. SIV is highly contagious, and proper animal care includes routine vaccination to prevent infectious diseases such as SIV. Accordingly, administration of a composition can be performed before, during, or after the occurrence of SIV. Treatment initiated before exposure to SIV or the development of SIV may result in decreased risk of infection or reduced severity of the signs of SIV if infection occurs. Treatment initiated after the development of SIV may result in decreasing the severity of the signs of SIV, or completely removing the signs. In this aspect of the invention, an “effective amount” is an amount effective to prevent the manifestation of signs of SIV, decrease the severity of the signs of SIV, and/or completely remove the signs.

The invention is defined in the claims. However, below there is provided a non-exhaustive listing of non-limiting exemplary aspects. Any one or more of the features of these aspects may be combined with any one or more features of another example, embodiment, or aspect described herein.

Aspect 1 is a genetically engineered Pichinde virus comprising: three ambisense genomic segments, wherein the first genomic segment comprises a coding region encoding a Z protein and a coding region encoding an L RdRp protein, wherein the second genomic segment comprises a coding region encoding a nucleoprotein (NP) and a second coding region, wherein the second coding region encodes at least one SIV protein, and wherein the third genomic segment comprises a coding region encoding a glycoprotein and a third coding region, wherein the third coding region encodes at least one SIV protein. Aspect 2 is the virus of any of Aspects 1 or 3-14 wherein the at least one SIV protein encoded by the second coding region is different than the at least one SIV protein encoded by the third coding region.

Aspect 3 is the virus of any of Aspects 1-2 or 4-14 wherein the at least one SIV protein encoded by the second coding region is the same as the at least one SIV protein encoded by the third coding region.

Aspect 4 is the virus of any of Aspects 1-3 or 5-14 wherein the second coding region encodes a polycistronic message encoding at least two SIV proteins.

Aspect 5 is the virus of any of Aspects 1˜4 or 6-14 wherein the third coding region encodes a polycistronic message encoding at least two SIV proteins.

Aspect 6 is the virus of any of Aspects 1-5 or 7-14 wherein the SIV proteins encoded by the second coding region are different than the SIV proteins encoded by the third coding region.

Aspect 7 is the virus of any of Aspects 1-6 or 8-14 wherein the SIV proteins encoded by the second coding region are the same as the SIV proteins encoded by the third coding region.

Aspect 8 is the virus of any of Aspects 1-7 or 9-14 wherein the second coding region expresses a monocistronic message encoding at least two SIV proteins, wherein the monocistronic message comprises nucleotides encoding a self-cleaving peptide, and wherein the nucleotides are located between the SIV proteins.

Aspect 9 is the virus of any of Aspects 1-8 or 10-14 wherein the third coding region expresses a monocistronic message encoding at least two SIV proteins, wherein the monocistronic message comprises nucleotides encoding a self-cleaving peptide, and wherein the nucleotides are located between the SIV proteins.

Aspect 10 is the virus of any of Aspects 1-9 or 11-14 wherein the self-cleaving peptide comprises a 2A peptide.

Aspect 11 is the virus of any of Aspects 1-10 or 12-14 wherein the 2A peptide comprises a P2A peptide, a T2A peptide, a E2A peptide, or a F2A peptide.

Aspect 12 is the virus of any of Aspects 1-11 or 13-14 wherein the at least one SIV protein of the second genomic segment and the at least one SIV protein of the third genomic segment are selected from SIV hemagglutinin (HA) proteins.

Aspect 13 is the virus of any of Aspects 1-12 or 14 wherein the at least two SIV proteins of the second genomic segment and the at least two SIV proteins of the third genomic segment are selected from SIV HA proteins.

Aspect 14 is the virus of any of Aspects 1-13 wherein the HA proteins are selected from a HA expressed by a SIV subtype H1N1, a HA expressed by a SIV subtype H1N2, or a HA expressed by a SIV subtype H3N2.

Aspect 15 is an infectious virus particle comprising the three genomic segments of any of Aspect 1-14.

Aspect 16 is a composition comprising an isolated infectious virus particle of Aspect 15.

Aspect 17 is a collection of vectors comprising: a first vector encoding the first genomic segment of any of Aspects 1-14, wherein the first genomic segment is antigenomic, a second vector encoding the second genomic segment of any of Aspects 1-14, wherein the second genomic segment is antigenomic, and a third vector encoding the third genomic segment of any of Aspects 1-14, wherein the third genomic segment is antigenomic Aspect 18 is the collection of Aspect 17 wherein the vectors are plasmids.

Aspect 19 is the collection of Aspects 17 or 18 wherein the plasmids further comprise a T7 promoter.

Aspect 20 is a method for making a genetically engineered Pichinde virus comprising: introducing into a cell the collection of vectors of any of Aspects 17-19; and incubating the cells in a medium under conditions suitable for expression and packaging of the first, second, and third genomic segments.

Aspect 21 is the method of Aspect 20 further comprising isolating an infectious virus particle from the medium.

Aspect 22 is the method any of Aspects 20 or 21 wherein the cells express a T7 polymerase.

Aspect 23 is an isolated infectious virus particle produced by the method of any of Aspects 20-22.

Aspect 24 is a composition comprising the isolated infectious virus particle of any of Aspect 23.

Aspect 25 is a reverse genetics system for making a genetically engineered virus comprising three vectors, wherein a first vector encodes the first genomic segment of any of any of Aspects 1-14, wherein the first genomic segment is antigenomic, wherein the second vector encodes the second genomic segment of any of any of Aspects 1-14, wherein the second genomic segment is antigenomic, and wherein the third vector encodes the third genomic segment of any of any of Aspects 1-14, wherein the third genomic segment is antigenomic.

Aspect 26 is the reverse genetics system of any of Aspect 25 wherein each vector comprises a T7 promoter.

Aspect 27 is a method for using a reverse genetics system, comprising: introducing into a cell the three vectors of genomic segments of any of Aspects 25 or 26; and incubating the cell under conditions suitable for the transcription of the three genomic segments and expression of the coding regions of each genomic segment.

Aspect 28 is the method of Aspect 27 further comprising isolating infectious virus particles produced by the cell, wherein each infectious virus particle comprises the three genomic segments.

Aspect 29 is the method of any of Aspects 27 or 28 wherein the introducing comprises transfecting a cell with the three vectors of genomic segments.

Aspect 30 is the method of any of Aspects 27-29 wherein the introducing comprises contacting the cell with an infectious virus particle comprising the three genomic segments.

Aspect 31 is the method of any of Aspects 27-30 wherein the cell is ex vivo.

Aspect 32 is the method of any of Aspects 27-31 wherein the cell is a vertebrate cell.

Aspect 33 is the method of any of Aspects 27-32 wherein the vertebrate cell is a mammalian cell.

Aspect 34 is the method of any of Aspects 27-33 wherein the mammalian cell is a porcine cell.

Aspect 35 is a method for producing an immune response in a subject, comprising: administering to a subject the infectious virus particle of Aspect 15 or the composition of Aspect 16.

Aspect 36 is the method of Aspect 35 wherein the subject is a vertebrate.

Aspect 37 is the method of any of Aspects 35 or 36 wherein the vertebrate is a mammal.

Aspect 38 is the method of any of Aspects 35-37 wherein the mammalian is a porcine animal.

Aspect 39 is the method of any of Aspects 35-38 wherein the immune response comprises a humoral immune response.

Aspect 40 is the method of any of Aspects 35-39 wherein the immune response comprises a cell-mediated immune response.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Immunogenicity and Protective Efficacy of a Recombinant Pichinde Viral-Vectored Vaccine Expressing Influenza Virus Hemagglutinin Antigen in Pigs

ABSTRACT: Influenza A virus of swine (IAV-S) is an economically important swine pathogen. The IAV-S hemagglutinin (HA) surface protein is the main target for vaccine development. In this study, we evaluated the feasibility of using the recombinant tri-segmented Pichinde virus (rPICV) as a viral vector to deliver HA antigen to protect pigs against IAV-S challenge. Four groups of weaned pigs (T01-T04) were included in the study. T01 was injected with PBS to serve as a non-vaccinated control. T02 was inoculated with rPICV expressing green fluorescence protein (rPICV-GFP). T03 was vaccinated with rPICV expressing the HA antigen of the IAV-S H3N2 strain (rPICV-H3). T04 was vaccinated with the recombinant HA protein antigen of the same H3N2 strain. Pigs were vaccinated twice at day 0 and day 21 and challenged at day 43 by intra-tracheal inoculation with the homologous H3N2 IAV-S strain. After vaccination, all pigs in T03 and T04 groups were seroconverted and exhibited high titers of plasma neutralizing antibodies. After challenge, high levels of IAV-S RNA were detected in the nasal swabs and bronchioalveolar lavage fluid of pigs in T01 and T02 but not in the T03 and T04 groups. Similarly, lung lesions were observed in T01 and T02, but not in the T03 and T04 groups. No significant difference in terms of protection was observed between the T03 and T04 group. Collectively, our results demonstrate that the rPICV-H3 vectored vaccine elicited protective immunity against IAV-S challenge. This study shows that rPICV is a promising viral vector for the development of vaccines against IAV-S.

### INTRODUCTION

Influenza A virus of swine (IAV-S) is one of the most important respiratory pathogens of swine [1]. The virus is widespread worldwide, causing significant economic loss to swine producers [2]. Clinically, pigs infected with IAV-S often display signs of an acute respiratory disease that rapidly resolves after 7 or 10 days of infection. However, when associated with other pathogens of the porcine respiratory disease complex, IAV-S infection can often lead to severe pneumonia [3]. There are three known major subtypes of IAV-S affecting pigs: H1N1, H1N2, and H3N2. Based on phylogenetic analysis of the IAV-S hemagglutinin (HA) sequences, the H1 and H3 subtypes can be further classified into multiple clades or lineages that are genetically distant from one another [4-7]. New strains of influenza virus frequently emerge in swine herds [8]. Genetic diversity of IAV-S represents a major hurdle for developing an efficacious vaccine [9-11].

Different platforms have been considered and used towards the development of vaccines against IAV [12]. Polyvalent, whole-inactivated virus (WIV) vaccines are commonly used for the control of IAV-S infection [14]. The WIV vaccines are found to be effective in protecting vaccinated pigs against antigenically matched IAV-S strains, but vaccine efficacy dramatically decreases when vaccinated pigs are exposed to antigenically mismatched virus strains [15-17]. Additionally, the use of WIV vaccines may enhance the severity of clinical outcomes if the vaccinated pigs are subsequently exposed to mismatched IAV-S strains of the same subtype [16,18], a phenomenon known as vaccine-associated enhanced respiratory disease (VAERD). Recently, a live-attenuated virus (LAV) vaccine against IAV-S was licensed for clinical application [19,20]. Experimental data demonstrate that the LAV vaccine could confer better heterologous protection than WIV vaccines [21-23]. However, reassortment between LAV and field IAV-S isolates has been documented, raising a real concern regarding the safety of the LAV vaccine platform [24].

IAV-S hemagglutinin (HA) is a target for the development of IAV-S vaccines. Different forms of the HA antigen have been tested in pigs as subunit vaccines, including HA protein—and DNA-based vaccines as well as viral vector-based vaccines expressing HA [25-30]. Several viral vectors have been employed to deliver HA antigen in pigs that include but are not limited to replication-defective human adenovirus type 5 (Ad5) and Orf virus [31,32].

Described herein is the development of Pichinde virus (PICV) as a viral vector to deliver HA antigen in pigs. PICV is an enveloped RNA virus within the Arenaviridae family. The virus was first isolated from rice rats (Oryzomys albigularis) in Colombia, South America [33]. PICV is considered non-pathogenic as there are no known PICV-associated diseases in humans or other animals [34]. The virus can infect a wide range of cell types from diverse host animal species that may include but are not necessarily limited to human, mouse, monkey, and avian species [34-37]. The wide host ranges and avirulent nature of PICV makes it a promising viral vector for flexible delivery of vaccine immunogens in humans and various animal species.

A reverse genetics system has recently been developed to generate recombinant trisegment PICV (rPICV) that carries as its genome two different small genomic(S) segments that encode the nucleoprotein (NP) and glycoprotein precursor complex (GPC), respectively, in each of the S segments and a long (L) segment that encodes the Z matrix protein and the L RNA-dependent RNA polymerase (RdRp). Additionally, the genome of the tri-segmented rPICV has been engineered to accommodate up to two foreign genes that can be used as vaccine antigens [34]. For instance, both HA and NP genes of a laboratory-adapted human IAV strain PR8 have been simultaneously inserted into the rPICV genome to produce a rPICV vectored vaccine that express IAV HA and NP concomitantly. When administered into mice, this experimental vaccine induces high levels of HA-specific neutralizing antibodies and NP-specific T cell response and protects the mice against a lethal challenge with IAV PR8 infection [34]. Another rPICV vectored vaccine expressing turkey arthritis reovirus antigens has also been generated and shown to induce high serum neutralizing antibody titers compared with the nonimmunized turkey poults [36].

In the present study, a rPICV vectored vaccine expressing the HA antigen of an IAV-S H3N2 strain (designated rPICV-H3) was generated. High levels of the H3 antigen were expressed in different pig cell types that were infected with rPICV-H3. Pigs vaccinated with the rPICV-H3 vaccine developed high titers of neutralizing antibody and were protected against a challenge infection with the homologous IAV-S H3N2 strain. Collectively, this study provides a proof of concept that rPICV can successfully be used as a viral vector for anti-influenza vaccination in swine.

### Materials and Methods

### Cells, Reagents, and Viruses

Baby hamster kidney (BHK-21) and porcine kidney-15 (PK-15) cells were cultured in complete Dulbecco's Modified Eagle Medium (DMEM) (Life Technologies, Grand Island, NY, USA), supplemented with 10% fetal bovine serum (FBS, Sigma-Aldrich, St. Louis, MO, USA) and 1× penicillin-streptomycin (100 units/mL of penicillin and 100 microg/mL of streptomycin, Life Technologies, Grand Island, NY, USA). Porcine alveolar macrophages (PAMs) were collected from lung lavage of pigs between 4 and 8 weeks old and were cryopreserved in a cell freezing medium containing 40% Roswell Park Memorial Institute (RPMI) (Life Technologies, Grand Island, NY, USA), 50% FBS, and 10% dimethyl sulfoxide. When needed, the cells were revived and cultured in complete RPMI supplemented with 10% FBS and 1× penicillin-streptomycin. Madin-Darby Canine Kidney (MDCK) cells were cultured in DMEM supplemented with 10% FBS, 1× penicillin-streptomycin, 0.2% Bovine serum albumin (BSA, Sigma-Aldrich, Saint Louis, MO, USA), and 25 mM HEPES (Hyclone, Life Technologies, Grand Island, NY, USA). All cell cultures were incubated at 37 C in a humidified environment containing 5% CO2.

The IAV-S strain A/swine/Texas/4199-2/1998 (H3N2 TX98) was obtained from The National Veterinary Services Laboratories (NVSL, Ames, IA, USA). The virus was propagated in MDCK cells in a virus inoculation medium (DMEM) supplemented with 1× penicillin-streptomycin, 0.2% BSA, 25 mM HEPES, and 1 microg/mL TPCK-treated trypsin (ThermoFisher Scientific, Rockford, IL, USA).

### Experimental Vaccines

The recombinant HA protein of H3N2 TX98 (designated H3-protein) was expressed in a baculovirus expression system via a contract with Genscript (Piscataway, NJ, USA). The purified protein was emulsified in 20% (v/v) Emulsigen-DL 90 (Phibro Animal Health Corporation, Omaha, NE, USA). Each dose of this vaccine contained 100 micrograms protein in a total volume of 2 mL.

The rPICV expressing the HA antigen of H3N2 TX98 was constructed as previously described [34]. Briefly, the HA sequence was codon-optimized for optimal protein expression in pigs using a commercial DNA synthesis service (Genscript, Piscataway, NJ, USA). The gene was cloned into both S1 and S2 of the rPICV reverse genetics system [34,35]. The three plasmids containing S1, S2, and L segments were co-transfected into BSRT7 cells to rescue the rPICV expressing H3 antigen (designated rPICV-H3). The rPICV-H3 vaccine was amplified in BHK-21 cells, and viral titers were quantified by conventional plaque assay [34]. A rPICV expressing green fluorescence protein (rPICV-GFP) that was generated previously was included in this study to serve as a vector control [34].

### Antigen Expression In Vitro

BHK-21, PK-15, or PAMs cultured separately in 24-well plates were infected with rPICV-H3 and rPICV-GFP at a multiplicity of infection (MOI) of 1 pfu per cell. At 48 h post-infection (hpi), cells were washed once with PBS and fixed in 500 microliters cold solution of methanol/acetone (1:1 v/v), followed by incubation with a monoclonal antibody specific to H3 antigen (generated in our laboratory) for 1 h at room temperature (RT). The cells were washed three times with phosphate buffer saline (PBS, pH 7.4) and incubated with a donkey anti-mouse Alexa flour 488 IgG H+L (Invitrogen, Life Technology corporation, Eugene, OR, USA) for 1 h at RT. After another three washes with PBS, fluorescent signals from cells were observed via an inverted fluorescence microscope. Fluorescent images were taken using the Nikon Eclipse Ts2R-FL operated by Nikon NIS Elements (ver 5.02). Cells infected with rPICV-GFP were directly visualized under fluorescence microscope 48 hpi. All images were taken using a 20× objective.

### Animal Experiment

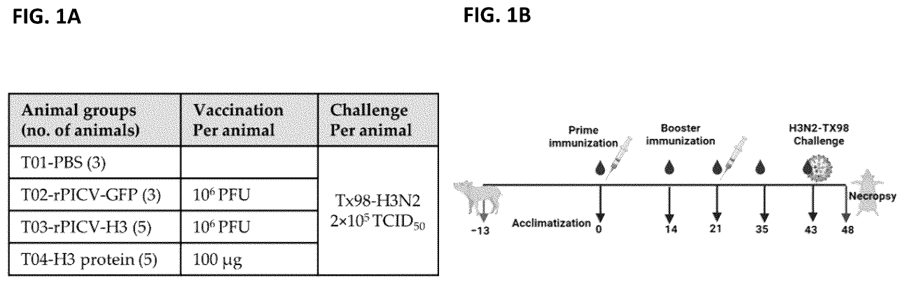

Sixteen 4-week-old pigs seronegative for porcine reproductive and respiratory syndrome virus (PRRSV) and IAV-S were purchased from the University of Nebraska-Lincoln (UNL) research farm and were housed in the animal biosafety level 2 (ABSL2) research facility at UNL. Pigs were randomly assigned into 4 treatment groups (T01-T04). T01 had three pigs that were injected intramuscularly (IM) with 2 mL PBS to serve as non-vaccinated controls. T02 had 3 pigs that were injected IM with 106 pfu of rPICV-GFP in 2 mL. Pigs in T01 and T02 groups were commingled in the same room throughout the course of the study. T03 contained five pigs that were vaccinated IM with 2 mL vaccine formulation containing 106 pfu of rPICV-H3. T04 had five pigs that were vaccinated IM with 2 mL of the H3-protein subunit vaccine. The pigs were vaccinated twice on days 0 and 21 (FIG. 1). One pig from T03 and another from T04 were humanely euthanized and removed from the study for welfare purposes due to injury during the blood collection process and for other reasons unrelated to rPICV infection, respectively.

Whole-blood samples with ethylenediaminetetraacetic acid (EDTA) anticoagulant were collected from all pigs at different time points before and after immunization and plasma samples were isolated to measure the humoral immune response.

On day 43 post vaccination (pv), all pigs were challenged by an intra-tracheal inoculation with 2×10⁵ TCID₅₀ of the H3N2 TX98. Nasal swabs were taken from all pigs daily post-challenge (pc) to measure potential IAV-S shedding. Pigs were humanely euthanized on day 48 pv (5 days post-challenge). Lung gross lesion was scored during necropsy by a veterinary pathologist who was blinded to the experimental treatment groups. Bronchioalveolar lavage fluid (BALF) samples were collected in cold PBS to measure viral genome copy number and infectivity. Samples of lung were fixed in 10% buffered formalin and processed by routine procedures for histopathologic examination as described in more details below.

### Serological Assay

Antibody responses against IAV-S nucleoprotein (NP) were measured by the Iowa State University Diagnostic Laboratory using a commercially available blocking ELISA (IDEXX, Montpellier, France). Results are expressed as test sample to negative control optical density (OD) ratios (S/N ratio). Samples with S/N ratio below 0.6 was considered positive, as it indicated the presence of higher levels of anti-NP antibodies in the test sample than in the negative control used in the assay.

Antibody responses against the H3 protein and the GFP were measured using ELISA. Briefly, flat-bottom 96-well plates were coated overnight at 4 C with 100 microliters of either the H3 protein or the GFP diluted in PBS to the final concentration of 2 microg/mL or 5 microg/mL, respectively. After washing with PBS containing 0.1% tween 20 (PBS-T20), the wells were blocked with 250 microliters/well of a blocking buffer (10% skim milk in PBS-T20) for 2 h at RT. Plasma samples were diluted in an assay buffer (5% skim milk in PBS-T20), and 100 microliters of diluted sample was added to each well of the plates in duplicate, followed by 1 h incubation at RT. After three washes with PBS-T20, 100_L of HRP-labeled goat anti-pig IgG (Sera Care, Milford, MA, USA) diluted 1:5000 in assay buffer was added to each well followed by 30 min incubation at room temperature. After three washes with PBS-T20, 100 microliters of ABTS substrate (Sera Care, Milford, MA, USA) was added to each well. The plate was incubated at RT between 5 and 25 min (until color was observed in the positive control wells). The reaction was stopped by adding 100 microliters of 1% sodium dodecyl sulfate (1% SDS) diluted in distilled water. Optical density (OD) was measured at 405 nm wavelength by an ELISA plate reader (Bio-Tek ELx808). For the H3 ELISA, samples were diluted 2-fold serially before adding to the ELISA plates. An arbitrary cutoff value equivalent to mean plus five standard deviations of the OD values of plasma samples from the non-immunized control animals was calculated. Antibody titers were determined at the highest dilutions with OD values above the cutoff value. For GFP-ELISA, plasma samples were tested at the dilution 1:100 and data are presented as the OD₄₀₅ value of each sample at the 1:100 dilution.

Virus neutralization assay and hemagglutination inhibition assay were essentially performed as previously described [38].

### Quantification of Viral Load

RNA was extracted from nasal swabs and BALF samples using Quick RNA viral Kit (Zymo Research, Costa Mesa, CA, USA) according to the manufacturer's protocol. Viral genomic copies were quantified using a real-time reverse transcription PCR (RT-PCR) by VetMax-Gold SIV Detection Kit (Life Technologies, Austin, TX, USA), as previously described [38]. Chemically synthesized RNA fragment with known copy numbers was used to establish a standard curve based on which the absolute copy numbers of viral RNA in each sample was estimated. Viral loads in nasal swabs were reported as log₁₀ copies per microliter of RNA used for the RT-PCR reaction. For statistical purposes, samples with undetectable levels of viral RNA were assigned a zero value.

Infectious IAV-S titers in BALF samples were determined by virus titration assay in MDCK cells cultured in 96-well plates. Briefly, the samples were diluted 10-fold serially in the virus inoculation medium, and 100 microliters of each dilution was inoculated in each well for a total of four wells per dilution. An immunofluorescence assay (IFA) was performed at 48 h to visualize the infected cells, and virus titer was calculated following the Reed-Muench method for estimating fifty percent endpoint [39].

### Lung Pathological Analysis

Lung gross lesions were evaluated during necropsy by a veterinary pathologist who was blinded to the treatment groups. The percentage of lung consolidation was calculated as described previously [40]. Sections of three different lung lobes, cranial, middle, and caudal, were stained with hematoxylin and eosin (H&E) following a routine pathological procedure. The slides were evaluated by a veterinary pathologist who was blinded to treatment groups following the scoring parameters as described previously [22]. Each of the three lung lobes was scored for six different parameters to generate a composite score ranging from 0 to 22 as previously described [22].

RNA in situ hybridization (ISH) was performed in the lung sections as previously described [38]. Composite scoring for the ISH slides was performed similarly to the composite score for microscopic lesions.

### Statistical Analysis

All statistical analyses were carried out using the GraphPad Prism 9.0. Antibody titers were log 2 transformed and analyzed using the mixed-effects analysis multiple comparisons. Gross lung lesion score, lung microscopic lesion score, viral genome copies, and virus titers were analyzed by ordinary one-way analysis of variance (ANOVA), followed by Tukey's multiple comparison test.

### Results

rPICV Vector Expressed High Levels of Gene-of-Interest in Pig Cells

We first evaluated the expression of the gene-of-interest (either HA antigen or GFP) in two types of pig cells: PK-15 and PAMs. BHK-21, which is routinely used to propagate rPICV, was included in the study for comparative purposes. The cells were infected with either rPICV-H3 or rPICV-GFP at an MOI of 1 pfu per well of the 24-well plate. At 48 hpi, GFP expression was observed in all three types of cells: BHK-21, PK-15, and PAM. An IFA was performed to detect HA antigen in cells infected with rPICV-H3 virus. HA-positive cells were observed in all three cell types tested. Collectively, the results indicate that rPICV can deliver genes-of-interest into pig cells.

Pigs Inoculated with PICV-GFP Do Not Transmit the Virus to Contact Pigs

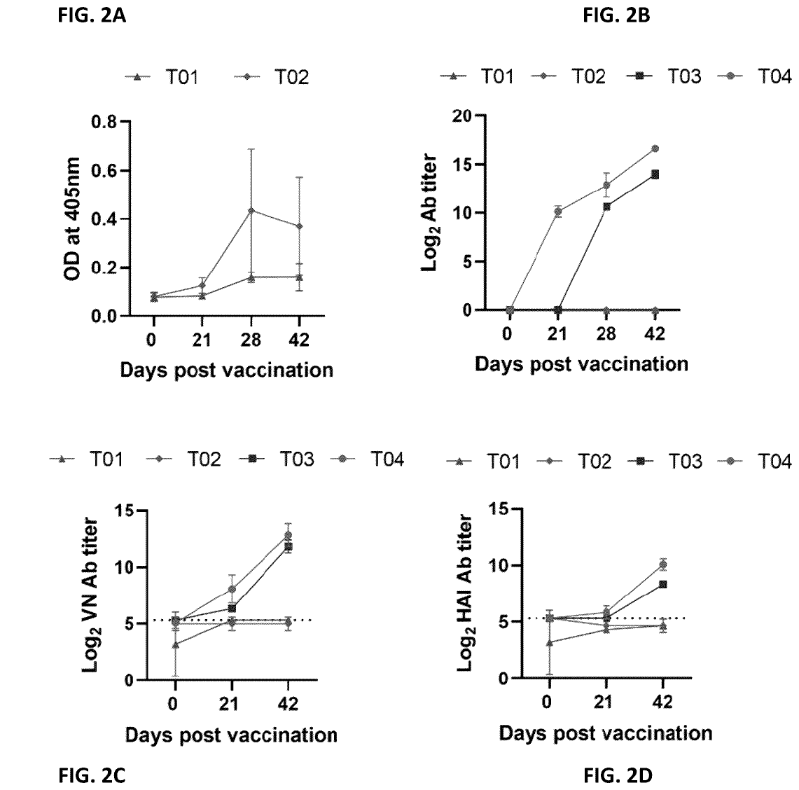

To determine whether rPICV can spread horizontally from one pig to another, the pigs that were injected with rPICV-GFP (T02) were commingled with the PBS-injected group (T01) throughout the course of the study. Antibodies against GFP were measured in plasma samples collected at multiple time points before and after vaccination using an indirect ELISA. Anti-GFP antibodies were detected in the T02 group starting from day 28 pv but not in group T01 (FIG. 2A). The result suggests that pigs vaccinated with rPICV-GFP did not transmit the virus to induce an anti-GFP antibody response in the contact pigs.

Pigs Immunized with rPICV-H3 or H3-Protein Subunit Vaccines Mounted a Robust Antibody Response

Antibodies specific to the H3 antigen were measured by an indirect ELISA. As expected, H3-specific antibodies were only detected in T03 and T04, not in the T01 and T02 groups (FIG. 2B). H3-specific ELISA antibodies were detected in T04 pigs starting from day 14 pv and continued to increase on days 28 and 35 pv. H3-specific ELISA antibodies were not detected in T03 pigs until day 28 pv, corresponding to day 7 post-boost. The results indicate that the H3-protein subunit vaccine elicited faster antibody responses than the rPICV-H3 vectored vaccine. However, no significant difference in the H3-specific ELISA antibody titers were observed between T03 and T04 after day 28 pv (corresponding to day 7 post-boost).

Next, virus neutralization (VN) and hemagglutination inhibition (HI) antibody titers were evaluated against H3N2 TX98, the homologous strain from which the HA gene was derived to generate the H3-protein and rPICV-H3 viral vectored vaccine used in this study. Background levels of VN and HI antibody titers (approximately 1:40) were observed in all pigs before vaccination and were maintained at similar titers in the T01 and T02 groups throughout the study (FIG. 2C, 2D). In contrast, VN and HI antibody titers significantly increased in the T03 and T04 groups, starting from day 21 pv, and further increased at day 42 pv. VN and HI antibody titers were not significantly different between the T03 and T04 groups at day 42 pv (FIG. 2C, 2D). The results indicate that both the rPICV-H3 vectored and H3-protein vaccines were able to elicit neutralizing antibodies in pigs.

Pigs Immunized with rPICV-H3 and H3-Protein Vaccines Were Protected against Challenge with the Homologous H3N2 IAV-S Strain

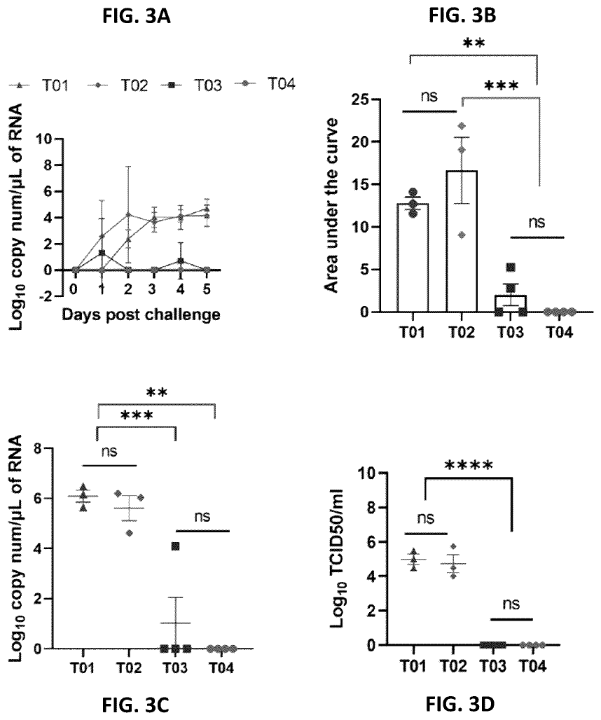

All pigs were challenged by an intra-tracheal inoculation with the H3N2 TX98 strain at day 43 pv (corresponding to day 22 post-boost). Nasal swabs were collected daily to measure IAV-S shedding. Viral RNA was detected in nasal swabs of all pigs in the T01 and T02 groups, starting from day 1 pc. In contrast, IAV-S RNA was not detected in any of the pigs in the T04 group at any sampling days (FIG. 3A). For group T03, two pigs had detectable levels of IAV-S RNA at one sampling day while the remaining two pigs did not have any detectable levels of viral RNA at any sampling days. To compare the total levels of IAV-S shedding among treatment groups, area under the curve (AUC) was calculated for individual pigs for the course of 5 days post challenge with IAV-S. The AUC of the T03 group was similar as that of the T04 group and was significantly lower than that of T01 and T02 (FIG. 3B).

All pigs were humanely sacrificed at day 5 post challenge, and a BALF sample was collected to measure IAV-S RNA by using an RT-PCR. High levels of IAV-S RNA were detected in the BALF from all pigs in T01 and T02 (FIG. 3C). In contrast, no IAV-S RNA was detected in the BALF from any of the pigs in group T04. One out of four pigs in the T03 group had a detectable level of IAV-S RNA in the BALF. This pig also had IAV-S RNA in its nasal swab collected on day 4 pc. To determine IAV-S infectivity, samples of BALF were titrated on MDCK cells. All BALF samples collected from T01 and T02 had infectious IAV-S with the titers ranging from 104.0 to 105.75 TCID₅₀/mL. In contrast, infectious IAV-S was not detected in the BALF of any pigs in T03 and T04, even though one pig in the T03 group had a detectable level of IAV-S RNA as determined by RT-PCR (FIG. 3D).

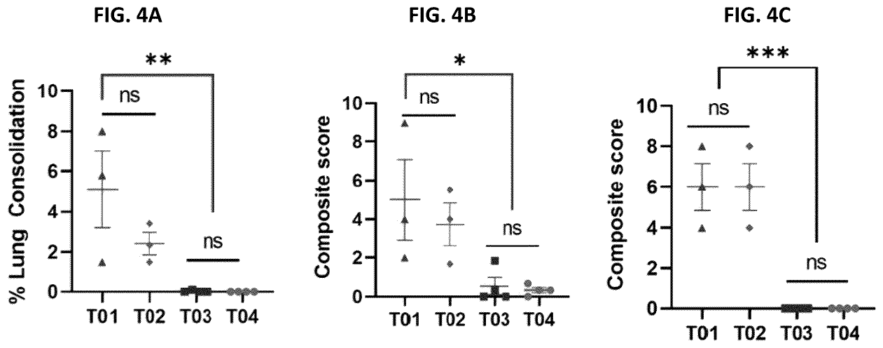

At necropsy, gross lung lesions were evaluated. Mild lung consolidation was observed in all pigs in the T01 and T02 groups, with the percentage of consolidated lung surface varying from 1.5% to 8%. Pigs in the T03 and T04 groups did not exhibit any visible lung consolidation (FIG. 4A).

During necropsy, samples from three lung lobes (apical, middle, and caudal) were selected for microscopic evaluation. Average composite scores of the three lobes are reported (FIG. 4B). Lung sections from the T01 and T02 groups exhibited mild-to-moderate interstitial pneumonia with necrotizing bronchiolitis and peribronchiolar lymphocytic cuffing. The mean composite microscopic scores of the T01 and T02 groups ranged from 1.7 to 9. On the contrary, no significant microscopic lesions were observed in the lung sections of pigs in the T03 and T04 groups (FIG. 4B).

In situ hybridization (ISH) was used to detect IAV-S NP gene transcript in the cardiac (middle) lung lobe of all pigs. NP transcripts were detected in all lung sections of pigs in the T01 and T02 groups. Conversely, no viral NP transcripts were observed in any lung section of pigs in the T03 and T04 groups (FIG. 4C).

Collectively, the results demonstrate that both the rPICV-H3 or H3-protein vaccines protected pigs from the challenge infection with a homologous H3N2 strain.

Pigs Immunized with H3-Protein Vaccine or rPICV-H3 Vectored Vaccine Tested Negative for IAV-S NP Antibodies by a Commercial ELISA Kit

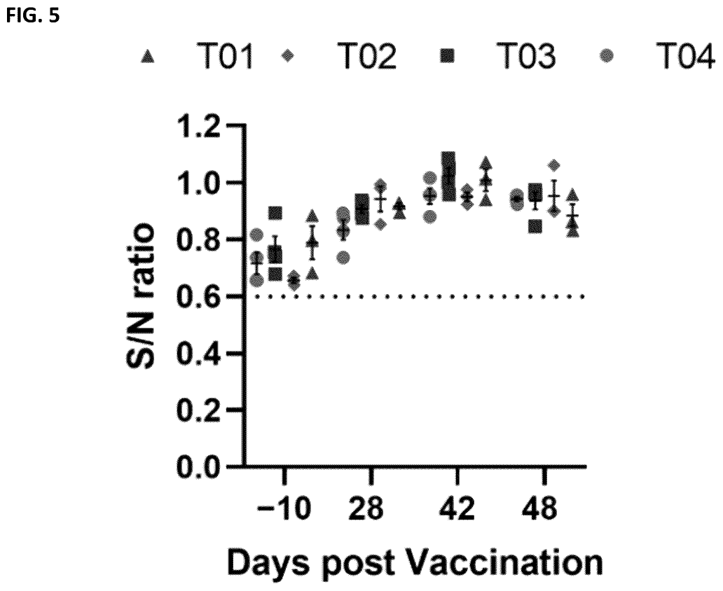

Antibodies specific to IAV-S NP were measured using a commercial ELISA kit that is routinely used for IAV-S serodiagnosis. All pigs tested negative for IAV-S NP antibody before vaccination (day-10) and continued to be negative till the end of the study (day 48 pv). The results demonstrate that the experimental vaccines used in this study did not induce antibody against IAV-S NP protein. Thus, vaccination with H-3 protein or rPICV-H3 vectored vaccine does not interfere with the serodiagnosis of IAV-S(FIG. 5).

### DISCUSSION

Immunization of pigs with a purified HA protein expressed in a mammalian expression system resulted in complete protection against homologous virus challenge [25]. However, pigs vaccinated with HA-protein subunit vaccine exhibited VAERD when they were challenged with an antigenically mismatched strain [44]. On the other hand, vaccination with a replication-defective Ad5 viral vector expressing HA did not result in VAERD upon challenge with an antigenically mismatched IAV-S strain [45]. These data suggest using a viral vector to deliver HA antigen is an attractive approach for the development of IAV-S vaccines.

An objective of this study was to evaluate the immunogenicity and protective efficacy of an rPICV viral vector expressing HA antigen of the IAV-S H3N2 strain in pigs (rPICV-H3). PICV is a non-pathogenic virus that was originally isolated from rats [33]. The viral genome has been engineered to allow it to carry up to two different vaccine immunogens. It has been demonstrated previously that rPICV viral vector effectively delivers vaccine immunogens in mice and turkeys [34,36]. In the present study, we demonstrated that pigs immunized with the rPICV-H3 vector vaccine mounted a robust humoral immune response and were completely protected against challenge infection with the homologous IAV-S H3N2 strain.

Pathogenesis of PICV infection in pigs has not been studied yet. There is no information pertaining to the target tissue and cell types for PICV replication, routes, and duration of viral shedding. Hence, we were interested in determining whether the pigs infected with rPICV spread the virus. For this purpose, pigs vaccinated with rPICV-GFP (T02) were commingled with those injected with PBS (T01). The rationale is that if pigs vaccinated with rPICV-GFP shed the virus, the naïve contact pigs (injected with PBS) would develop antibodies against GFP. Anti-GFP antibodies were detected in pigs vaccinated with rPICVGFP but not in the contact pigs. Thus, our preliminary data suggest that pigs vaccinated with rPICV do not spread the Pichinde virus horizontally to the contact pigs.

It has been well-documented that vaccination of pigs with an HA-protein-based vaccine will result in complete protection [25]. Therefore, we included a group of pigs that were immunized with a purified H3-protein vaccine (T04) in this study for comparative purposes. As expected, pigs vaccinated with the H3-protein vaccine developed a robust humoral antibody response, which can be detected at day 21 pv and continued to increase post boost. On the contrary, H3-specific antibodies were only detected at day 28 pv, corresponding to day 7 post boost in pigs vaccinated with rPICV-H3 vectored vaccine (T03). Although pigs in the T03 group developed antibody responses later than those in the T04 group, their VN—and HAI-antibody titers sharply increased and reached similar titers compared to those in the T04 group by day 42 pv. In previous studies, the amount of protein mass used to vaccinate pigs ranged from 0.5 micrograms to 25 micrograms per pig per immunization, which were sufficient to confer sterilizing immunity against the challenge infection with a homologous H1N1 strain [25,26,44]. In this study, we immunized pigs with 100 micrograms purified H3 protein, which was significantly higher than the antigenic dose used in previous studies [44]. We believe that the earlier antibody response observed in pigs immunized with the H3-protein vaccine was partially due to the higher antigenic mass used to vaccinate pigs.

None of the pre-vaccination samples had antibodies against IAV-S NP as determined by a commercial ELISA (FIG. 5). Similarly, none of the pre-vaccination samples had antibodies against HA protein of H3N2 TX98 (FIG. 2B). However, samples collected prior to the study exhibited some level of HAI- and VN-antibody titers against H3N2 TX98 challenge strain (titer less than or equal to 1:40, equivalent to 5.32 Log₂). VN—and HAI-titers maintained the same or slightly declined levels by day 42 pv in pigs injected with PBS (T01) or with rPICV-GFP (T02). In contrast, these antibody titers rapidly increased in pigs vaccinated with the H3-protein subunit vaccine (T04) or rPICV-H3 vectored vaccine (T03). Thus, the VN—and HAI-antibody titer in samples collected pre-study might be due to non-specific and/or cross-reactivity. It was reported recently that antisera from SARS-COV-2 patients could cross-react with the IAV HA protein [46]. The pigs used in this study were unlikely to be infected with SARS-COV-2. Instead, they might be pre-exposed to one of the porcine coronaviruses, which are widely prevalent in swine herds [47].

After challenge infection, viral RNA was not detected in nasal swabs, BALF, and lung section of pigs in the T04 group. Thus, under the experimental conditions of this study, the H3-protein vaccine conferred sterilizing immunity against the homologous H3N2 strain. Two pigs in T03 exhibited detectable levels of IAV-S RNA in their nasal swabs at one sampling day and one of these two pigs had detectable levels of viral RNA in its BALF sample at day 5 post challenge. When titrated in MCDK cells, the BALF sample in the T03 pig that contained IAV-S RNA, as determined by RT-PCR, did not show evidence of successful IAV-S infection, indicating that the BALF sample was non-infectious or at levels below the limit of detection of the assay. Statistically, the viral IAV-S RNA copies in nasal swabs and BALF were not significantly different between the T03 and T04 groups. Likewise, the lung lesion scores were not different between the T03 and T04 groups, both in terms of gross- and microscopic-lesion. Collectively, the results suggest that the rPICV-H3 vectored vaccine confers an equivalent level of protection as compared to the H3-protein vaccine.

ELISA kits detecting antibodies specific to the NP are available for the serodiagnosis of IA V-S. All pigs in the T03 and T04 groups tested negative by the NP ELISA kit throughout the course of the study, even though they exhibited high antibody titers against the HA protein. Thus, pigs vaccinated with the H3-protein vaccine or with the rPICV-H3 vectored vaccine can be serologically distinguished from those that are naturally infected with IAV-S by NP ELISA. Therefore, the H3-protein subunit and rPICV-H3 vectored vaccine used in this study fulfills the requirement of a DIVA (differentiating infected from vaccinated animals) vaccine. It should be noted that antibodies against the IAV-S NP protein were not detected in pigs in the T01 and T02 groups at day 5 post challenge infection (corresponding to day 48 pv). We believed that this was too soon after infection for pigs in the T01 and T02 groups to mount an antibody response to the NP protein. Indeed, anti-NP antibodies were only detected in a few pigs that were experimentally inoculated with H1 and H3 IAV-S at day 14 post infection [48].

### Conclusions

rPICV is a promising viral vector for the development of IAV-S vaccines. These results demonstrate that the rPICV vectored vaccine does not spread horizontally among pigs. Importantly, pigs vaccinated with the rPICV-H3 vectored vaccine induced HAI and VN antibodies and were fully protected against challenge infection with a homologous IAV-S strain.

### Citations for Example 1

1. Rajao, D. S.; Anderson, T. K.; Gauger, P. C.; Vincent, A. L. Pathogenesis and Vaccination of Influenza A Virus in Swine. Curr. Top Microbiol. Immunol. 2014, 385, 307-326.
2. Haden, C.; Painter, T.; Fangman, T.; Holtkamp, D. J. Assessing Production Parameters and Economic Impact of Swine Influenza, PRRS and Mycoplasma hyopneumoniae on Finishing Pigs in a Large Production System. In Proceedings of the American Association of Swine Veterinarians Annual Meeting, Denver, CO, USA, 10-13 Mar. 2012. Available online: https://vetmed.iastate.edu/sites/default/files/vdpam/Cara % 20Haden %20AASV %20Abstract. pdf (accessed on 14 Jul. 2022).
3. Vincent, A. L.; Lager, K. M.; Anderson, T. K. A Brief Introduction to Influenza A Virus in Swine. Methods Mol. Biol. 2014, 1161, 243-258.
4. Anderson, T. K.; Macken, C. A.; Lewis, N. S.; Scheuermann, R. H.; Van Reeth, K.; Brown, I. H.; Swenson, S. L.; Simon, G.; Saito, T.; Berhane, Y.; et al. A Phylogeny-Based Global Nomenclature System and Automated Annotation Tool for H1 Hemagglutinin Genes from Swine Influenza A Viruses. MSphere 2016, 1, e00275-16.
5. Bolton, M. J.; Abente, E. J.; Venkatesh, D.; Stratton, J. A.; Zeller, M.; Anderson, T. K.; Lewis, N. S.; Vincent, A. L. Antigenic Evolution of H3N2 Influenza a Viruses in Swine in the United States from 2012 to 2016. Influenza Other Respir. Viruses 2019, 13, 83-90.
6. Walia, R. R.; Anderson, T. K.; Vincent, A. L. Regional Patterns of Genetic Diversity in Swine Influenza a Viruses in the United States from 2010 to 2016. Influenza Other Respir. Viruses 2019, 13, 262-273.
7. Souza, C. K.; Anderson, T. K.; Chang, J.; Venkatesh, D.; Lewis, N. S.; Pekosz, A.; Shaw-Saliba, K.; Rothman, R. E.; Chen, K. F.; Vincent, A. L. Antigenic Distance between North American Swine and Human Seasonal H3N2 Influenza A Viruses as an Indication of Zoonotic Risk to Humans. J. Virol. 2022, 96, e0137421. Vaccines 2022, 10, 1400 13 of 14
8. Zeller, M. A.; Li, G.; Harmon, K. M.; Zhang, J.; Vincent, A. L.; Anderson, T. K.; Gauger, P. C. Complete Genome Sequences of Two Novel Human-Like H3N2 Influenza A Viruses, A/swine/Oklahoma/65980/2017 (H3N2) and A/Swine/Oklahoma/65260/2017 (H3N2), Detected in Swine in the United States. Microbiol. Resour. Announc. 2018, 7, e01203-18.
9. Anderson, T. K.; Nelson, M. I.; Kitikoon, P.; Swenson, S. L.; Korslund, J. A.; Vincent, A. L. Population dynamics of cocirculating swine influenza A viruses in the United States from 2009 to 2012. Influenza Other Respir. Viruses 2013, 7, 42-51.
10. Steel, J.; Lowen, A. C. Influenza A Virus Reassortment. In Influenza Pathogenesis and Control-Volume I; Compans, R., Oldstone, M., Eds.; Current Topics in Microbiology and Immunology; Springer: Cham, Swizterland, 2014; Volume 385, pp. 377-401.
11. Ma, W. Swine influenza virus: Current status and challenge. Virus Res. 2020, 288, 198118.
12. Chen, J.; Wang, J.; Zhang, J.; Ly, H. Advances in Development and Application of Influenza Vaccines. Front. Immunol. 2021, 12, 711997.
13. Khatri, M.; Dwivedi, V.; Krakowka, S.; Manickam, C.; Ali, A.; Wang, L.; Qin, Z.; Renukaradhya, G. J.; Lee, C. W. Swine influenza H1N1 virus induces acute inflammatory immune responses in pig lungs: A potential animal model for human H1N1 influenza virus. J. Virol. 2010, 84, 11210-11218.
14. Sandbulte, M. R.; Spickler, A. R.; Zaabel, P. K.; Roth, J. A. Optimal Use of Vaccines for Control of Influenza A Virus in Swine. Vaccines 2015, 3, 22-73.
15. Vincent, A. L.; Ciacci-Zanella, J. R.; Lorusso, A.; Gauger, P. C.; Zanella, E. L.; Kehrli, M. E.; Janke, B. H.; Lager, K. M. Efficacy of inactivated swine influenza virus vaccines against the 2009 A/H1N1 influenza virus in pigs. Vaccine 2010, 28, 2782-2787.
16. Vincent, A. L.; Lager, K. M.; Janke, B. H.; Gramer, M. R.; Richt, J. A. Failure of protection and enhanced pneumonia with a US H1N2 swine influenza virus in pigs vaccinated with an inactivated classical swine H1N1 vaccine. Vet. Microbiol. 2008, 126, 310-323.
17. Kitikoon, P.; Gauger, P. C.; Anderson, T. K.; Culhane, M. R.; Swenson, S.; Loving, C. L.; Perez, D. R.; Vincent, A. L. Swine influenza virus vaccine serologic cross-reactivity to contemporary US swine H3N2 and efficacy in pigs infected with an H3N2 similar to 2011-2012 H3N2v. Influenza Other Respir. Viruses 2013, 7, 32-41.
18. Gauger, P. C.; Vincent, A. L.; Loving, C. L.; Lager, K. M.; Janke, B. H.; Kehrli, M. E.; Roth, J. A. Enhanced pneumonia and disease in pigs vaccinated with an inactivated human-like (delta-cluster) H1N2 vaccine and challenged with pandemic 2009 H1N1 influenza virus. Vaccine 2011, 29, 2712-2719.
19. Richt, J. A.; Lekcharoensuk, P.; Lager, K. M.; Vincent, A. L.; Loiacono, C. M.; Janke, B. H.; Wu, W. H.; Yoon, K. J.; Webby, R. J.; Solorzano, A.; et al. Vaccination of pigs against swine influenza viruses by using an NS1-truncated modified live-virus vaccine. J. Virol. 2006, 80, 11009-11018.
20. Lee, J.; Li, Y.; Li, Y.; Cino-Ozuna, A. G.; Duff, M.; Lang, Y.; Ma, J.; Sunwoo, S.; Richt, J. A.; Ma, W. Bat influenza vectored NS1-truncated live vaccine protects pigs against heterologous virus challenge. Vaccine 2021, 39, 1943-1950.
21. Loving, C. L.; Lager, K. M.; Vincent, A. L.; Brockmeier, S. L.; Gauger, P. C.; Anderson, T. K.; Kitikoon, P.; Perez, D. R.; Kehrli, M. E. Efficacy in Pigs of Inactivated and Live Attenuated Influenza Virus Vaccines against Infection and Transmission of an Emerging H3N2 Similar to the 2011-2012 H3N2v. J. Virol. 2013, 87, 9895-9903.
22. Gauger, P. C.; Loving, C. L.; Khurana, S.; Lorusso, A.; Perez, D. R.; Kehrli, M. E., Jr.; Roth, J. A.; Golding, H.; Vincent, A. L. Live attenuated influenza A virus vaccine protects against A (H1N1) pdm09 heterologous challenge without vaccine associated enhanced respiratory disease. Virology 2014, 471-473, 93-104.
23. Abente, E. J.; Rajao, D. S.; Santos, J.; Kaplan, B. S.; Nicholson, T. L.; Brockmeier, S. L.; Gauger, P. C.; Perez, D. R.; Vincent, A. L. Comparison of Adjuvanted-Whole Inactivated Virus and Live-Attenuated Virus Vaccines against Challenge with Contemporary, Antigenically Distinct H3N2 Influenza A Viruses. J. Virol. 2018, 92, e01323-18.
24. Sharma, A.; Zeller, M. A.; Li, G.; Harmon, K. M.; Zhang, J.; Hoang, H.; Anderson, T. K.; Vincent, A. L.; Gauger, P. C. Detection of live attenuated influenza vaccine virus and evidence of reassortment in the U.S. swine population. J. Vet. Diagn. Investig. 2020, 32,301-311.
25. Loeffen, W. L.; de Vries, R. P.; Stockhofe, N.; van Zoelen-Bos, D.; Maas, R.; Koch, G.; Moormann, R. J.; Rottier, P. J.; de Haan, C. A. Vaccination with a soluble recombinant hemagglutinin trimer protects pigs against a challenge with pandemic (H1N1) 2009 influenza virus. Vaccine 2011, 29, 1545-1550.
26. Vander Veen, R.; Kamrud, K.; Mogler, M.; Loynachan, A. T.; McVicker, J.; Berglund, P.; Owens, G.; Timberlake, S.; Lewis, W.; Smith, J.; et al. Rapid development of an efficacious swine vaccine for novel H1N1. PLOS Curr. 2009, 1, RRN1123.
27. Fynan, E. F.; Webster, R. G.; Fuller, D. H.; Haynes, J. R.; Santoro, J. C.; Robinson, H. L. DNA vaccines: Protective immunizations by parenteral, mucosal, and gene-gun inoculations. Proc. Natl. Acad. Sci. USA 1993, 90, 11478-11482.
28. Larsen, D. L.; Karasin, A.; Olsen, C. W. Immunization of pigs against influenza virus infection by DNA vaccine priming followed by killed-virus vaccine boosting. Vaccine 2001, 19, 2842-2853.
29. Said, A.; Lange, E.; Beer, M.; Damiani, A.; Osterrieder, N. Recombinant equine herpesvirus 1 (EHV-1) vaccine protects pigs against challenge with influenza A (H1N1) pmd09. Virus Res. 2013, 173, 371-376.
30. Xu, J.; Huang, D.; Liu, S.; Lin, H.; Zhu, H.; Liu, B.; Lu, C. Immune responses and protection efficacy of a recombinant swinepox virus expressing HA against swine H3N2 influenza virus in mice and pigs. Virus Res. 2012, 167, 188-195.
31. Wesley, R. D.; Tang, M.; Lager, K. M. Protection of weaned pigs by vaccination with human adenovirus 5 recombinant viruses expressing the hemagglutinin and the nucleoprotein of H3N2 swine influenza virus. Vaccine 2004, 22, 3427-3434. Vaccines 2022, 10, 1400 14 of 14
32. Joshi, L. R.; Knudsen, D.; Pineyro, P.; Dhakal, S.; Renukaradhya, G. J.; Diel, D. G. Protective Efficacy of an Orf Virus-Vector Encoding the Hemagglutinin and the Nucleoprotein of Influenza A Virus in Swine. Front. Immunol. 2021, 12, 747574.
33. McLay, L.; Liang, Y.; Ly, H. Comparative analysis of disease pathogenesis and molecular mechanisms of NewWorld and Old World arenavirus infections. J. Gen. Virol. 2014, 95 Pt 1, 1-15.
34. Dhanwani, R.; Zhou, Y.; Huang, Q.; Verma, V.; Dileepan, M.; Ly, H.; Liang, Y. A Novel Live Pichinde Virus-Based Vaccine Vector Induces Enhanced Humoral and Cellular Immunity after a Booster Dose. J. Virol. 2015, 90, 2551-2560.
35. Dhanwani, R.; Ly, H.; Liang, Y. Recombinant Tri-Segmented Pichinde Virus as a Novel Live Viral Vaccine Platform. Methods Mol. Biol. 2017, 1581, 169-179.
36. Kumar, P.; Sharafeldin, T. A.; Kumar, R.; Huang, Q.; Liang, Y.; Goyal, S. M.; Porter, R. E.; Ly, H.; Mor, S. K. Development of a Recombinant Pichinde Virus-Vectored Vaccine against Turkey Arthritis Reovirus and Its Immunological Response Characterization in Vaccinated Animals. Pathogens 2021, 10, 197.
37. Brisse, M.; Huang, Q.; Rahman, M.; Di, D.; Liang, Y.; Ly, H. RIG-I and MDA5 Protect Mice From Pichinde Virus Infection by Controlling Viral Replication and Regulating Immune Responses to the Infection. Front. Immunol. 2021, 12, 801811.
38. Sun, H.; Sur, J. H.; Sillman, S.; Steffen, D.; Vu, H. L. X. Design and characterization of a consensus hemagglutinin vaccine immunogen against H3 influenza A viruses of swine. Vet. Microbiol. 2019, 239, 108451.
39. Reed, L. J.; Muench, H. A simple method of estimating fifty percent endpoints. Am. J. Epidemiol. 1938, 27, 493-497.
40. Halbur, P. G.; Paul, P. S.; Frey, M. L.; Landgraf, J.; Eernisse, K.; Meng, X. J.; Lum, M. A.; Andrews, J. J.; Rathje, J. A. Comparison of the pathogenicity of two US porcine reproductive and respiratory syndrome virus isolates with that of the Lelystad virus. Vet. Pathol. 1995, 32, 648-660.
41. Rossman, J. S.; Lamb, R. A. Influenza virus assembly and budding. Virology 2011, 411, 229-236.
42. Dou, D.; Revol, R.; Ostbye, H.; Wang, H.; Daniels, R. Influenza A Virus Cell Entry, Replication, Virion Assembly and Movement. Front. Immunol. 2018, 9, 1581.
43. Sautto, G. A.; Kirchenbaum, G. A.; Ross, T. M. Towards a universal influenza vaccine: Different approaches for one goal. Virol. J. 2018, 15, 17.
44. Rajao, D. S.; Loving, C. L.; Gauger, P. C.; Kitikoon, P.; Vincent, A. L. Influenza A virus hemagglutinin protein subunit vaccine elicits vaccine-associated enhanced respiratory disease in pigs. Vaccine 2014, 32, 5170-5176.
45. Braucher, D. R.; Henningson, J. N.; Loving, C. L.; Vincent, A. L.; Kim, E.; Steitz, J.; Gambotto, A. A.; Kehrli, M. E., Jr. Intranasal vaccination with replication-defective adenovirus type 5 encoding influenza virus hemagglutinin elicits protective immunity to homologous challenge and partial protection to heterologous challenge in pigs. Clin. Vaccine Immunol. 2012, 19, 1722-1729.
46. Murugavelu, P.; Perween, R.; Shrivastava, T.; Singh, V.; Ahmad Parray, H.; Singh, S.; Chiranjivi, A. K.; Thiruvengadam, R.; Singh, S.; Yadav, N.; et al. Non-neutralizing SARS-COV-2 directed polyclonal antibodies demonstrate cross-reactivity with the HA glycans of influenza virus. Int. Immunopharmacol. 2021, 99, 108020.
47. Turlewicz-Podbielska, H.; Pomorska-Mol, M. Porcine Coronaviruses: Overview of the State of the Art. Virol. Sin. 2021, 36, 833-851.
48. Takemae, N.; Tsunekuni, R.; Uchida, Y.; Ito, T.; Saito, T. Experimental infection of pigs with H1 and H3 influenza A viruses of swine by using intranasal nebulization. BMC Vet. Res. 2018, 14, 115.

### Example 2

Assessment of immune responses to a trivalent Pichinde virus-vectored vaccine expressing hemagglutinin genes of three co-circulating Influenza A virus subtypes in pigs

ABSTRACT: Recombinant Pichinde virus (rPICV) is a non-pathogenic arenavirus that can infect a wide range of cell types from various animal species. Our previous study showed that pigs vaccinated with a recombinant PICV-vectored vaccine expressing the hemagglutinin (HA) gene of an H3N2 strain of influenza A virus of swine (IAV-S) generated virus-neutralizing antibodies and were protected against infection with the matching H3N2 strain. The objective of the current study was to evaluate the immunogenicity and protective efficacy of a trivalent PICV-vectored vaccine expressing HA antigens representing three co-circulating IAV-S subtypes in swine: H1N1, H1N2, and H3N2. Pigs immunized with the trivalent PICV vaccine developed virus-neutralizing (VN) and hemagglutination inhibition (HI) antibodies against all three IAV-S strains from which the HA genes were used to insert into the trivalent PICV vaccine. However, the HI titers were notably lower than the VN titers. Following a challenge with the H1N1 strain, pigs vaccinated with the trivalent vaccine had minimal copies of IAV-S RNA genomes detected in nasal swabs and bronchoalveolar lavage fluid. In contrast, the non-vaccinated control pigs showed high copies of IAV-S genome in these two types of samples. Overall, the results demonstrate that the trivalent rPICV-vectored vaccine elicits antibody responses against the three targeted IAV-S strains and provides protection against one of them in pigs. Therefore, PICV functions as a viral vector for effectively delivering multiple vaccine antigens in swine.

### INTRODUCTION

Influenza A virus in swine (IAV-S) is a major respiratory pathogen affecting swine production worldwide [1]. Typically, IAV-S infection results in acute respiratory diseases with mild clinical signs and low mortality rates [2]. However, IAV-S infection significantly reduces infected pigs' average daily weight gain and imposes a considerable economic burden on the swine industry [3,4]. Besides its economic impact, IAV-S poses a significant public health concern due to its zoonotic potential [5].

Three subtypes of IAV-S, H1N1, H1N2, and H3N2, are widespread in North America [6,7]. The H1 and H3 genes of IAV-S can be phylogenetically classified into lineages, which are further subdivided into clades. The percentage of pairwise nucleotide distances between the clades within a lineage can be as high as 16% [8-10]. The profound genetic diversity poses a significant challenge in developing broadly protective influenza vaccines (reviewed in [11-13]). Whole-inactivated virus (WIV) vaccines are widely used to control IAV-S. These vaccines contain multiple IAV-S isolates representing the IAV-S linages cocirculating in the field. Generally, WIV vaccines confer robust protection against homologous but not against antigenically mismatched IAV-S strains [14,15]. Moreover, pigs vaccinated with WIV vaccines might exhibit more severe clinical diseases when exposed to antigenically mismatched IAV-S strains, a phenomenon known as vaccine-associated enhanced

respiratory diseases (VAERD) [14]. Another limitation of WIV vaccines is that their efficacy is significantly reduced when administered to piglets with maternally derived antibodies (MDA) [16].

Live-attenuated influenza virus (LAV) vaccines are also commercially available. Unlike WIV vaccines, LAV vaccines confer partial protection against mismatched IAV-S isolates instead of inducing VAERD [17-20]. However, the LAV vaccine has the potential to revert to virulence after reassortment with field IAV-S [21].

Significant effort has been made to develop subunit vaccines targeting hemagglutinin (HA) protein, the surface glycoprotein responsible for viral attachment to the host cells. The HA protein-based vaccines elicit complete protection against homologous IAV-S strains [22]. However, the HA protein-based vaccine can induce VAERD, like WIV vaccines, when the vaccinated pigs are exposed to mismatched IAV-S strains [23].

DNA vaccines based on the HA gene have been tested in pigs. Immunization of pigs with a naked DNA plasmid results in weak immune responses, primarily due to ineffective cellular entry of the plasmid. Hence, various approaches, including in vivo electroporation and needle-free intradermal applicators, have been devised to enhance the immunogenicity of DNA plasmid vaccines [24-29]. Typically, hemagglutinin inhibition (HI) antibody titers are not detected in DNA-vaccinated pigs after one immunization dose. Moreover, pigs vaccinated with the DNA vaccines are not protected from developing severe lung lesions upon challenge infection with the homologous IAV-S strain. However, they may have shorter durations and lower magnitudes of virus shedding in their nasal cavity.

Self-replicating RNA replicon systems derived from positive-strand, single-stranded viral RNA genomes such as alphaviruses and pestiviruses have been utilized for delivering the HA genes to pigs [30-32]. In these systems, the structural genes of the alphaviruses or pestiviruses are replaced with genes encoding vaccine antigens, such as the HA gene of IAV-S. This way, the alphavirus or pestivirus nonstructural genes function as the RNA replication complex to amplify hybrid mRNA molecules. Pigs vaccinated with the self replicating RNA replicon vaccines containing the IAV-S HA genes are protected against challenge infection with homologous IAV-S strains [31,32].

Several viral vectors, including replication-defective adenovirus, pseudorabies virus, and Orf virus have been utilized to deliver the HA gene [33-40]. In general, viral vector vaccines carrying the HA gene induce effective protective immune responses against the homologous IAV-S strain from which the HA gene was derived. For instance, intramuscular or intranasal vaccination of pigs with a replication-defective adenovirus type 5 (Ad5) encoding HA proteins has been shown to provide protective immunity against challenge infection from the homologous viral strains and partial protection against heterologous challenges [35-38].

Notably, pigs receiving the Ad5 viral vector or alphavirus RNA replicon vaccines do not develop VAERD after being challenged with a heterologous IAV-S strain [35]. These observations suggest that intracellular delivery of the HA antigen may help to prevent VAERD.

Pichinde virus (PICV), a nonpathogenic member of the Arenaviridae family, has a broad cell tropism and holds significant potential as a viral vector for vaccine advancement [41,42]. Recombinant PICV-vectored vaccines have been tested in mice, guinea pigs, and turkeys. In all cases, animals that received the PICV-vectored vaccines mounted robust immune responses against the vaccine antigens [41,42].

Example 1 demonstrates that a monovalent PICV-vectored vaccine expressing the HA gene of the H3N2 virus induced high titers of virus-neutralizing and hemagglutination inhibition antibodies and protected pigs against a subsequent challenge from the homologous H3N2 virus [43]. In the present study, we demonstrated that pigs vaccinated with a trivalent PICV expressing representative HA genes from three co-circulating IAV-S strains mounted robust immune responses against all three antigens, and were protected against challenge infection with the homologous H1N1 strain.

### Materials and Methods

### Cells, Reagents, and Viruses

Madin-Darby canine kidney (MDCK, ATCC CCL-34) and baby hamster kidney 21 (BHK-21, ATCC C-13) were obtained from the American type culture collection (ATCC, Manassas, VA, USA). MDCK cells were cultured in DMEM (Life Technologies, Grand Island, NY, USA) supplemented with 10% heat-inactivated fetal bovine serum (FBS, Sigma-Aldrich, St. Louis, MO, USA), 2.5% bovine serum albumin (BSA, Sigma-Aldrich, Saint Louis, MO, USA), and 25 mM HEPES (Hyclone, Life Technologies, Grand Island, NY, USA). BHK-21 cells were cultured in DMEM supplemented with 10% FBS. BSRT7 cells, which are BHK-21 cells stably expressing T7 RNA polymerase, were obtained from K. K. Conzelmann (Ludwig-Maximilians-Universität, Germany) and cultured in MEM supplemented with 10% FBS and 1 microgram/mL Geneticin (Life Technologies, Carlsbad, CA, USA).

Three IAV-S strains: A/swine/Iowa/A01202099/2011 (H1N1-pdm09), A/swine/Minnesota/A01392045/2013 (H1N2-deltala), and A/swine/Texas/4199-2/1998 (H3N2-TX98) were obtained from the National Veterinary Services Laboratories (NVSL, Ames, IA, USA). Virus stock for all three IAV-S were grown in MDCK cells.

### Vaccines Used in the Study

The whole-inactivated virus vaccine Flusure XP was purchased from Zoetis (Parsippany, NJ, USA). The vaccine was prepared and administered according to the label. The WIV contains four heat-killed IAV-S strains: A/Swine/Iowa/110600/2000 (H1N1), A/Swine/Oklahoma/0726H/2008 (H1N2), A/Swine/North Carolina/394/2012 (H3N2), and A/Swine/Minnesota/872/2012 (H3N2).

The recombinant PICV-vectored vaccine was prepared as previously described [41]. The HA genes of three representative IAV-S strains, H1N1-pdm09, H1N2-deltala, and H3N2-TX98, were codon-optimized for optimal protein expression in pigs and synthesized using a commercial DNA synthesis service (Genscript, Piscataway, NJ, USA). The HA gene from each of the three IAV-S strains was cloned into multiple cloning sites of both S1 and S2 plasmids and co-transfected into BSRT-7 cells along with a third plasmid encoding for L segment. Infectious recombinant PICV was rescued as previously described [41]. The resulting recombinant PICV modified to express the HA gene was plaque purified and amplified in BHK-21 cells. Virus titers were quantified using the conventional plaque assay in BHK-21 cells. The three PICVs expressing individual HA antigens were combined at a concentration of 106 PFU/mL for each PICV to make a cocktail of the trivalent PICV-vectored vaccine (TPV) used in animal vaccination.

### Vaccination and Challenge Experiment

The vaccination and challenge experiment were conducted in a weaned pig's model, as described previously [43]. Twenty-one 4-week-old pigs, seronegative for IAV-S and PRRSV, were obtained from Midwest Swine Research. The pigs were randomly assigned into four treatment groups and were accommodated in the animal biosafety level 2 (ABSL2) research facility at the University of Nebraska-Lincoln (UNL). Group 1 consisted of three pigs that were neither vaccinated nor challenged with IAV-S(NV/NC). Groups 2, 3, and 4 consisted of six pigs each. Group 2 was injected intramuscularly (i.m.) with 2 mL of PBS to serve as a non-vaccination control. Group 3 was vaccinated i.m. with the WIV vaccine following the manufacturer's instruction. Group 4 was inoculated i.m. with the TPV vaccine. The WIV and TPV vaccines were administered twice, with a three-week interval between injections.

Blood samples were collected from all the pigs on days 0, 21, and 42 post-vaccination (p.v.) and plasma from these samples was isolated and stored at −20° C. for later evaluation of the humoral immune response.

On day 42 post-vaccination (p.v.), pigs in groups 2 to 4were challenged with 2×10⁵ TCID₅₀ of the H1N1 pdm09 virus. The challenge virus was diluted in 4 mL of serum-free DMEM, with 2 mL administered via the intra-tracheal route and the remaining 2 mL given via the intra-nasal route, with 1 mL in each nostril. Pigs in group 1 were not subjected to the virus challenge and served as the no-vaccination/no-challenge (NV/NC) control.

Nasal swabs were collected daily post-challenge (p.c.) to measure potential IAV-S shedding. On day 5 p.c., all pigs were humanely euthanized. During necropsy, gross lung lesions were scored by a veterinary pathologist blinded to the treatment group. Bronchioalveolar lavage fluid (BALF) was collected in 50 mL of cold PBS to measure IAV-S RNA within the lungs. After that, lung tissue samples were collected and fixed in 10% buffered formalin to examine potential microscopic lesions.

### Immunological Assay

Antibodies specific to the IAV-S nucleoprotein (NP) were detected using a commercial blocking ELISA (IDEXX, Westbrook, ME, USA). A sample-to-negative (S/N) ratio smaller than 0.6 indicated the presence of anti-NP antibodies in the plasma sample.

Antibody responses against H1N1-pdm09, H1N2-deltala, and H3N2-TX98 viruses were measured using virus neutralization (VN) and hemagglutination inhibition (HI) assays [44]. The plasma samples were diluted 2-fold, serially after an initial dilution of 1:10. A titer of <1:10 was assigned the value of 5 for graphical representation purposes.

Virus neutralization against PICV was performed using plasma samples collected from TPV-vaccinated pigs on days 0, 21, and 42 p.v. The samples were diluted 2-fold, serially in DMEM in a 96-well plate and incubated with an equal volume containing 100 TCID₅₀ of recombinant PICV expressing green fluorescent protein (PICV-GFP) for 1 h at 37° C. After that, the plasma-virus mixture was transferred to a 96-well plate containing confluent BHK-21 cells seeded 24 h earlier. The plates were incubated for 48 h at 37° C. in a humidified atmosphere containing 5% CO2. The expression of GFP was visualized using a fluorescence microscope. Neutralization titers were defined as the reciprocal of the highest plasma dilution that showed complete inhibition of PICV-GFP infection.

### Quantification of Viral Load

IAV-S RNA extraction and quantification from nasal swabs and BALF samples were performed as previously described [43]. Viral loads were reported as log₁₀ copies of IAV RNA per 100 microliter of sample used to extract RNA. For statistical purposes, samples with undetectable levels of IAV RNA were assigned a value of 0.8, equivalent to the assay detection limit.

### Pathological Analysis of Lungs

Gross lung lesions were evaluated during necropsy by a veterinary pathologist who was blinded to the treatment groups. The percentage of lung consolidation was calculated as described previously [45]. Middle lung lobe sections were stained with hematoxylin and eosin (H & E), followed by a routine pathological procedure for histopathological evaluation [17].

To identify IAV-S infected cells within the tissue, in situ hybridization (ISH) targeting IAV-NP RNA was performed in sections of the middle lung lobe. Virus-infected cell frequency in the airway epithelium and pulmonary parenchyma was scored on a 5-point scale: 0 for no signals, 1 for minimal occasional signals, 2 for mild scattered signals, 3 for moderate scattered signals, and 4 for abundant signals.

### Statistical Analysis

All statistical analyses were carried out using GraphPad Prism 9.3.1 Antibody titers were log 2 transformed and analyzed using the mixed-effects analysis multiple comparisons. Gross lung lesion scores, lung microscopic lesion scores, and viral genome copies were analyzed using the Kruskal-Wallis test. Subsequently, the mean rank of each treatment group was compared with the mean rank of the PBS control group using the uncorrected Dunn's test.

### Results

Antibody Responses after Vaccination

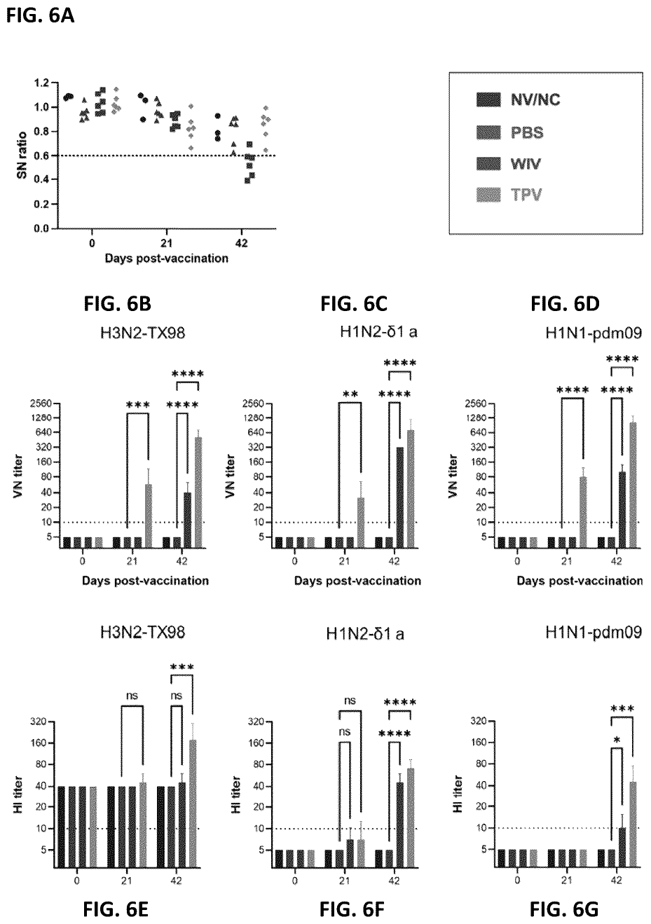

All plasma samples that were collected before vaccination tested negative for IAV-S exposure with the commercial ELISA kit that detects antibodies specific to the IAV-S NP. As expected, pigs vaccinated with the TPV vaccine were negative for anti-NP antibodies throughout the 42-day post-vaccination period. On the other hand, five of the six pigs vaccinated with the WIV vaccine developed detectable levels of anti-NP antibodies by day 42 p.v. (FIG. 6A).

Virus-neutralizing (VN) antibodies were measured against the three homologous IAV-S strains from which the HA genes were derived to generate the TPV vaccine. In the TPV group, VN antibodies against each of the three IAV-S subtypes were detected at day 21 p.v. and the neutralization titers increased after the booster immunization, reaching geometric mean titer between 1:533 and 1:1067 on day 42 p.v. (FIG. 6B-D). Pigs in the WIV group developed VN antibodies against the three IAV-S strains. However, VN titers in the WIV group were only detected on day 42 p.v. As expected, pigs in the PBS and NV/NC groups did not exhibit VN titers against the three IAV-S strains tested (FIG. 6B-D).

Next, we measured hemagglutination inhibition (HI) antibody titers against the three tested IAV-S strains. Notably, before vaccination, all pigs had a background HI titer of 1:40 against the H3N2 TX98 strain (FIG. 6E) even though these samples tested negative for the presence of antibodies against the IAV-S NP (FIG. 6A). On day 42 p.v., all six pigs in the TPV group had increased HI titers against the H3N2 TX98 virus. On the other hand, only one of the six pigs in the WIV group exhibited an increase in HI titers against the H3N2 TX98 virus, while none of the pigs in the NV/NC or PBS group showed any increase in HI titers against the H3N2 virus at day 42 p.v. (FIG. 6E).

Unlike the case of H3N2, plasma samples collected before vaccination did not show any detectable HI titers against the H1N2-deltala or H1N1-pdm09 strains (FIG. 6F,G). On day 42 p.v., all pigs in the TPV group developed HI antibodies against the H1N1-pdm09 and H1N2-deltala virus with a geometric mean titer of 1:45 and 1:71, respectively. For the WIV group, the mean HI titers of samples collected on day 42 p.v. against H1N1-pdm09 and H1N2-deltala viruses were 1:45 and 1:10, respectively. Pigs in the NV/NC or PBS groups did not show HI antibody titers against the H1N2-deltala and H1N1-pdm09 viruses at any sampling days (FIG. 6F,G).

### Viral Genomic RNA in Nasal Secretions and Lungs

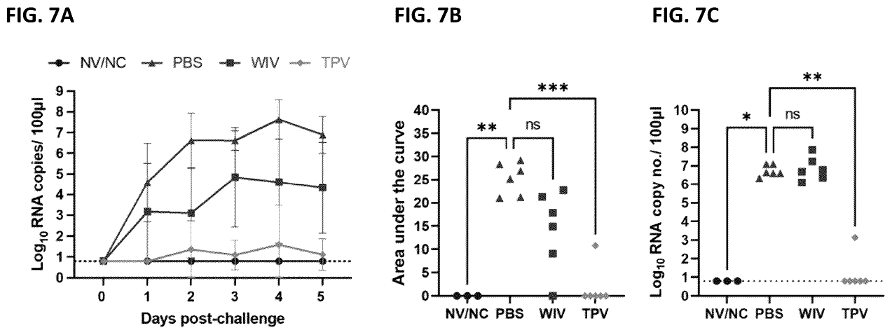

All pigs in the PBS group exhibited detectable levels of the IAV-S genomic RNA copies in their nasal swabs, starting from day 1 after challenge infection with the H1N1-pdm09. The IAV-S genomic RNA copies peaked on day 4 p.c., reaching the mean of 107.6 copies per 100 microliters of sample (FIG. 7A). Five of the six pigs in the WIV group had detectable IAV-S genomic RNA copies in their nasal secretions starting from day 1 p.c, while only one pig in the TPV group had relatively low copies IAV-S genomic RNA on days 2 and 4 p.c. (FIG. 7A).

The area under the curve (AUC) was calculated to assess the overall extent of IAV-S shedding across the 5-day observational period. The TPV group had a significantly smaller AUC than the PBS group (FIG. 7B). The WIV group had a comparatively smaller AUC than the PBS group, but this difference did not reach statistical significance. As expected, no AUC values were observed in the NV/NC group, because no IAV-S genomic RNA was detected in nasal swabs of those pigs.

On day 5 p.c., BALF samples were collected to quantify the IAV-S RNA genome copies present within the lungs. All pigs in the PBS and WIV groups had high IAV-S genomic RNA copies in their BALF samples (FIG. 7C). Conversely, only one pig in the TPV group had detectable IAV-S genomic RNA in its BALF sample. No IAV-S genomic RNA was detected in the BALF samples collected from pigs in the NV/NC group (FIG. 7C).

### Macroscopic and Microscopic Lung Lesions

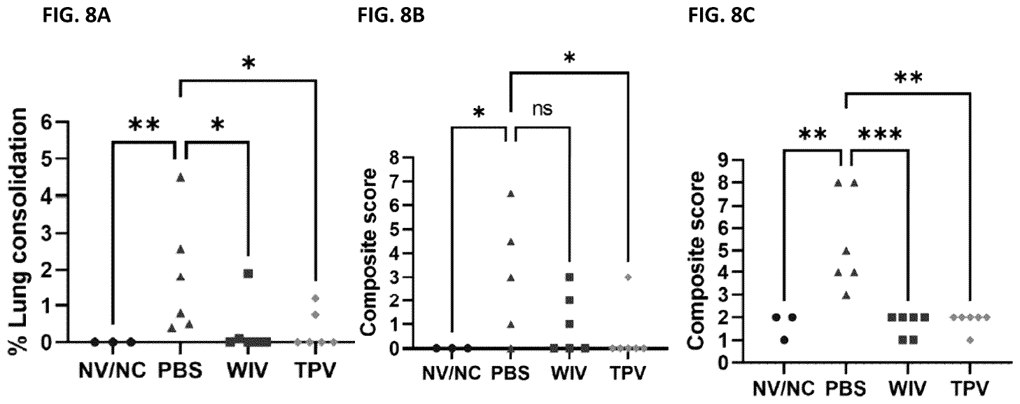

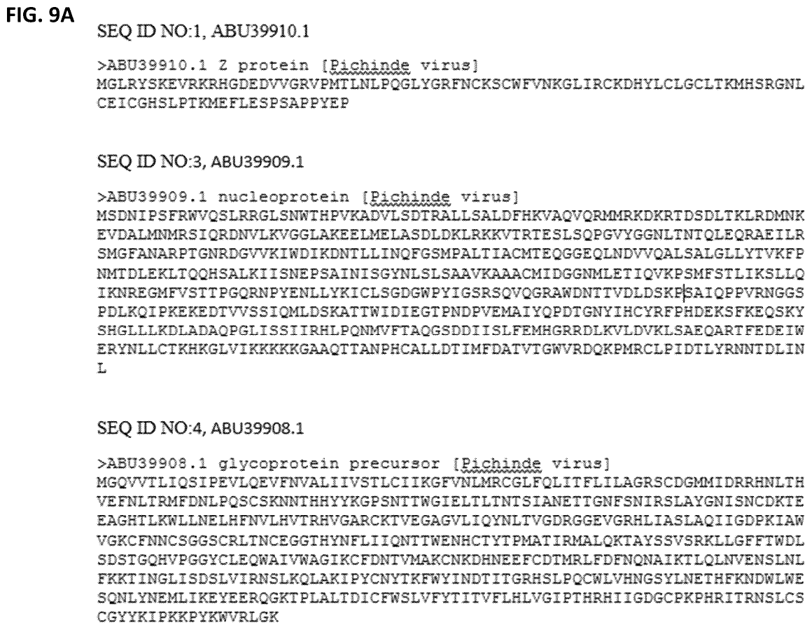

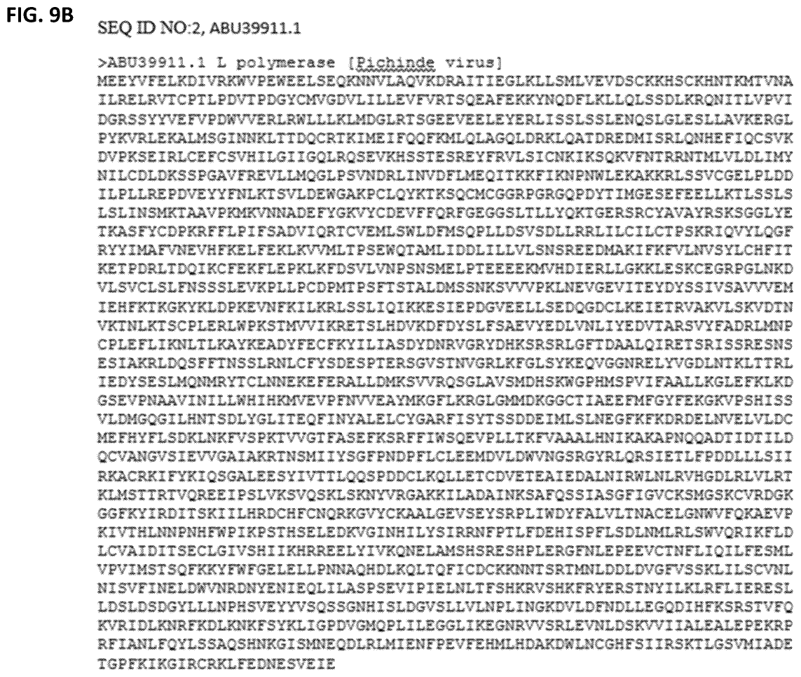

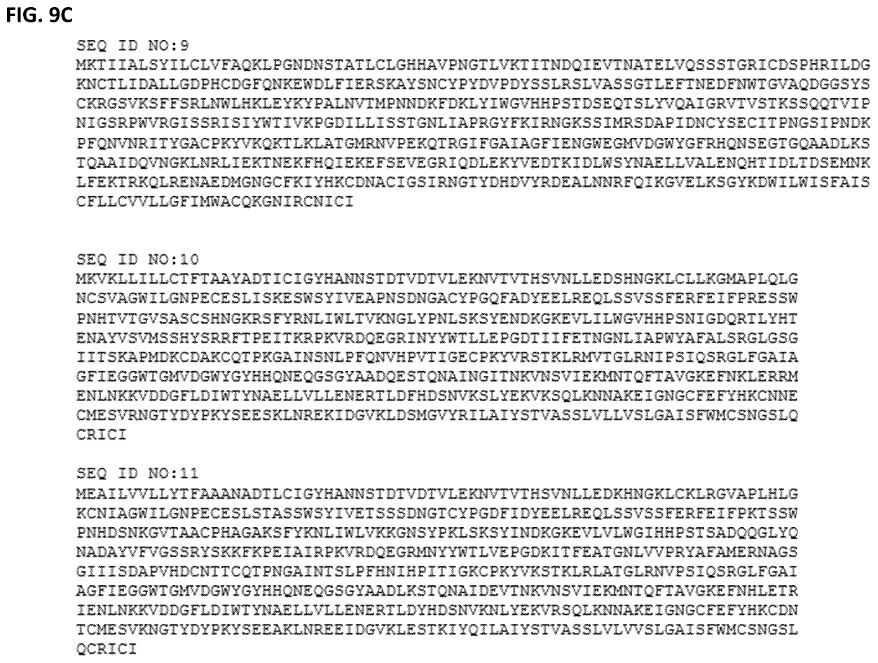

No pigs in the NV/NC group showed lung consolidation indicative of IAV-S infection. Conversely, all pigs in the PBS group displayed lung consolidation, varying from 0.5% to 4.5% (FIG. 8A). Only one pig from the TPV group and two from the WIV group exhibited lung consolidation. Both TPV and WIV groups demonstrated significantly lower levels of lung consolidation compared to the PBS group (FIG. 8A).

Pigs in the NV/NC group did not exhibit noticeable microscopic lung lesions (FIG. 8B). The lungs from the pigs in the PBS group presented mild peribronchiolar lymphocytic cuffing, necrosis of bronchiolar epithelial lining, and low levels of interstitial pneumonia, with the composite microscopic lung scores ranging between 0 and 9 (FIG. 8B). Three out of the six pigs in the WIV group displayed microscopic lesions, scoring between 1 and 3. Only one pig in the TPV group showed mild microscopic lesions. Consequently, the TPV group's microscopic lung lesion scores were statistically lower than those of the PBS group (FIG. 8B).

An in-situ hybridization (ISH) assay was utilized to detect IAV-S infected cells within the lung tissue. The lung samples from pigs in the PBS group displayed a high number of virus-infected cells, particularly within the bronchial and bronchiolar epithelium. Conversely, virus-infected cells were rarely detected in the lung tissues of pigs in the TPV or WIV group. As a result, both the TPV and WIV groups exhibited significantly lower SH scores compared to the PBS group (FIG. 8C).

### Antibody Responses Against the PICV Vector (Anti-Vectored Immunity)

Antibody responses against the viral vector could potentially hamper the vaccine's effectiveness. Therefore, we performed a virus-neutralization assay to detect PICV-specific neutralizing antibodies. For the virus-neutralization assay, we employed PICV-GFP, enabling real-time monitoring of the virus-infected cells. Plasma samples obtained from pigs vaccinated with TPV on days 0 and 21 p.v. did not display any inhibition of PICV-GFP infection in BHK-21 cells. This was evident from the comparable numbers of GFP-positive cells observed in wells treated with these samples and in control cells without any plasma. However, at a dilution of 1:4, samples collected on day 42 p.v. showed some neutralization effects against the virus but were insufficient to prevent infection completely. Together, the data demonstrated that pigs in the TPV group did not induce significant neutralizing antibodies against the viral vector.

### DISCUSSION

Three IAV-S subtypes —H1N1, H1N2, and H3N2— are co-circulating in U.S. swine herds. Due to the substantial genetic and antigenic differences, viruses within these three subtypes do not confer cross-protection against each other. Thus, we aimed to develop a trivalent PICV vectored vaccine containing representative HA genes of these three IAV-S subtypes to broaden the vaccine antigenic coverage. However, antigenic competition might occur when multiple vaccine antigens are combined within a vaccine. Thus, this study is primarily centered on evaluating the antibody responses in pigs immunized with the trivalent PICV vectored vaccine (TPV). Our results demonstrate that pigs receiving the TPV exhibited VN and HI antibodies at similar titers against each of the three IAV-S strains, from which the HA genes were utilized in developing the TPV vaccine. Furthermore, pigs vaccinated with the TPV vaccine were protected against challenge with the H1N1-pdm09 virus. Together, these results indicate that the trivalent PICV vaccine elicits a strong and non-interfering immunity against all three IAV-S subtypes.

In this study, we included a group of pigs vaccinated with the WIV for reference purposes. However, it is essential to note that a direct comparison of immune responses and protection outcomes between pigs vaccinated with the TPV vaccine and those vaccinated with the WIV vaccine is challenging, primarily due to differences in the antigenic composition of these two vaccines. The TPV vaccine contains HA genes that match the three IAV-S strains employed in the HI and VN assays. In contrast, the WIV vaccine includes four IAV-S strains genetically different from the three strains used in the VN and HI assays. Notably, the WIV vaccine lacks the H1N1-pdm09 strain, which explains the absence of detectable HI titers against this virus strain in WIV-vaccinated pigs. Following challenge infection with the H1N1-pdm09 virus, WIV-vaccinated pigs exhibited no statistically significant differences in viral loads in nasal swabs or BALF samples collected during necropsy compared to the PBS control group. Intriguingly, WIV-vaccinated pigs displayed less severe gross lesions than the PBS control group. Furthermore, very few IAV-S-infected cells were detected in lung sections of the WIV-vaccinated pigs. It is plausible that other IAV-S strains present in the WIV may induce partial cross-protective immunity against the H1N1-pdm09 strain.

Pigs inoculated with the WIV vaccine displayed detectable antibodies against the IAV-S nucleoprotein, whereas those administered with the TPV vaccine did not exhibit such antibodies. Consequently, it is possible to serologically differentiate TPV-vaccinated pigs from those naturally infected with IAV-S by employing a commercially available ELISA designed for detecting antibodies against the IAV-S NP. This serological distinction is important for disease control and eradication programs, as it enables the detection of naturally infected animals within the vaccinated population. This, in turn, allows for the implementation of appropriate measures, such as quarantine or culling of the infected herds, to effectively eliminate the viruses from the animal herd [46-48].

In this study, all pre-vaccination samples consistently displayed a baseline HI titer of 1:40 against the H3N2 virus. For pigs in the PBS or NV/NC group, a baseline HI titer of 1:40 against the H3N2 virus was observed throughout the study period. However, samples collected from pre-vaccination, or the PBS and NV/NC groups, did not exhibit antibodies against the IAV-S nucleoprotein, as determined with a commonly used commercial blocking ELISA for IAV-S serodiagnosis. Additionally, these samples did not show VN antibody titers against the H3N2 TX98 strain. We therefore concluded that the pigs used in this study had not been previously exposed to IAV-S, nor had they received maternally derived anti-IAV-S antibodies. We believe that the HI titers against the H3N2 virus in the pre-vaccination samples were likely the result of nonspecific inhibition. It is notable that the background HI titer was only observed against the H3N2 virus but not against the H1N1 or H1N2 viruses. We observed a similar pattern in a previous study, in which all pre-vaccination samples also displayed a baseline HI titer of 1:40 against the H3N2 TX98 strain [43]. Before performing the HI assay, all samples were treated with the receptor-destroying enzyme and pre-adsorbed to red blood cells to eliminate potential nonspecific inhibitors. Thus, the underlying reasons for the background HI titers against H3N2 TX98 observed in this study remain unknown.

Antibody responses directed against the viral vector can present a formidable obstacle in the development of viral vector vaccines. In the present study, no neutralizing antibodies against PICV were detected in samples collected on day 21 p.v. Samples collected after the booster immunization reduced PICV infection only at the lowest plasma dilution (1:4), indicating that the pigs did not mount a significant anti-vector antibody response. This finding is further supported by the observation that both VN and HI antibody titers against the three IAV-S strains significantly increased after the booster immunization. A prior study also demonstrated that mice and guinea pigs, when vaccinated with a PICV-vector vaccine expressing influenza virus antigens, exhibited significantly elevated antibody titers against the influenza virus upon receiving repeated immunizations [41]. Consequently, the absence of neutralizing antibody responses against the PICV vector makes it suitable for repeated immunization protocols.

### Conclusions

A trivalent PICV-vectored vaccine was developed that contains HA genes from three concurrently circulating IAV-S subtypes in the U.S. This trivalent vaccine effectively elicited VN and HI antibodies against all three representative IAV-S strains. Notably, pigs immunized with the trivalent PICV-vectored vaccine were fully protected against challenge infection with the H1N1-pdm09 strain, one of the three IAV-S strains whose HA gene was included in the PICV-vectored vaccine.

### Citations for Example 2

1. Vincent, A.; Awada, L.; Brown, I.; Chen, H.; Claes, F.; Dauphin, G.; Donis, R.; Culhane, M.; Hamilton, K.; Lewis, N.; et al. Review of Influenza A Virus in Swine Worldwide: A Call for Increased Surveillance and Research. Zoonoses Public Health 2014, 61, 4-17.
2. Janke, B. H. Influenza A virus infections in swine: Pathogenesis and diagnosis. Vet. Pathol. 2014, 51, 410-426.
3. Calderon Diaz, J. A.; Fitzgerald, R. M.; Shalloo, L.; Rodrigues da Costa, M.; Niemi, J.; Leonard, F. C.; Kyriazakis, I.; Garcia Manzanilla, E. Financial Analysis of Herd Status and Vaccination Practices for Porcine Reproductive and Respiratory Syndrome Virus, Swine Influenza Virus, and Mycoplasma hyopneumoniae in Farrow-to-Finish Pig Farms Using a Bio-Economic Simulation Model. Front. Vet. Sci. 2020, 7, 556674.
4. Haden, C.; Painter, T.; Fangman, T.; Holtkamp, D. J. Assessing Production Parameters and Economic Impact of Swine Influenza, PRRS and Mycoplasma Hyopneumoniae on Finishing Pigs in a Large Production System. In Proceedings of the American Association of Swine Veterinarians Annual Meeting, Denver, CO, USA, 10-13 Mar. 2012; Available online: vetmed.iastate.edu/sites/default/files/vdpam/Cara % 2520Haden %2520AASV %2520Abstrac t.pdf (accessed on 28 Nov. 2023).
5. Ma, W.; Kahn, R. E.; Richt, J. A. The pig as a mixing vessel for influenza viruses: Human and veterinary implications. J. Mol. Genet. Med. Int. J. Biomed. Res. 2008, 3, 158-166.
6. Walia, R. R.; Anderson, T. K.; Vincent, A. L. Regional patterns of genetic diversity in swine influenza A viruses in the United States from 2010 to 2016. Influenza Other Respir. Viruses 2018, 13, 262-273.
7. Anderson, T. K.; Campbell, B. A.; Nelson, M. I.; Lewis, N. S.; Janas-Martindale, A.; Killian, M. L.; Vincent, A. L. Characterization of co-circulating swine influenza A viruses in North America and the identification of a novel H1 genetic clade with antigenic significance. Virus Res. 2015, 201, 24-31.
8. Anderson, T. K.; Macken, C. A.; Lewis, N. S.; Scheuermann, R. H.; Van Reeth, K.; Brown, I. H.; Swenson, S. L.; Simon, G.; Saito, T.; Berhane, Y.; et al. A Phylogeny-Based Global Nomenclature System and Automated Annotation Tool for H1 Hemagglutinin Genes from Swine Influenza A Viruses. mSphere 2016, 1, e00275-16.
9. Anderson, T. K.; Chang, J.; Arendsee, Z. W.; Venkatesh, D.; Souza, C. K.; Kimble, J. B.; Lewis, N. S.; Davis, C. T.; Vincent, A. L. Swine Influenza A Viruses and the Tangled Relationship with Humans. Cold Spring Harb. Perspect. Med. 2021, 11, a038737.
10. Anderson, T. K.; Nelson, M. I.; Kitikoon, P.; Swenson, S. L.; Korslund, J. A.; Vincent, A. L. Population dynamics of cocirculating swine influenza A viruses in the United States from 2009 to 2012. Influenza Other Respir. Viruses 2013, 7, 42-51.
11. Salvesen, H. A.; Whitelaw, C. B. A. Current and prospective control strategies of influenza A virus in swine. Porc. Health Manag. 2021, 7, 23.
12. Mancera Gracia, J. C.; Pearce, D. S.; Masic, A.; Balasch, M. Influenza A Virus in Swine: Epidemiology, Challenges and Vaccination Strategies. Front. Vet. Sci. 2020, 7, 647.
13. Sandbulte, M. R.; Spickler, A. R.; Zaabel, P. K.; Roth, J. A. Optimal Use of Vaccines for Control of Influenza A Virus in Swine. Vaccines 2015, 3, 22-73.
14. Vincent, A. L.; Lager, K. M.; Janke, B. H.; Gramer, M. R.; Richt, J. A. Failure of protection and enhanced pneumonia with a US H1N2 swine influenza virus in pigs vaccinated with an inactivated classical swine H1N1 vaccine. Vet. Microbiol. 2008, 126, 310-323.
15. Lee, J. H.; Gramer, M. R.; Joo, H. S. Efficacy of swine influenza A virus vaccines against an H3N2 virus variant. Can. J. Vet. Res. 2007, 71, 207-212.
16. Vincent, A. L.; Ma, W. J.; Lager, K. M.; Richt, J. A.; Janke, B. H.; Sandbulte, M. R.; Gauger, P. C.; Loving, C. L.; Webby, R. J.; Garcia-Sastre, A. Live Attenuated Influenza Vaccine Provides Superior Protection from Heterologous Infection in Pigs with Maternal Antibodies without Inducing Vaccine-Associated Enhanced Respiratory Disease. J. Virol. 2012, 86, 10597-10605.
17. Gauger, P. C.; Loving, C. L.; Khurana, S.; Lorusso, A.; Perez, D. R.; Kehrli, M. E., Jr.; Roth, J. A.; Golding, H.; Vincent, A. L. Live attenuated influenza A virus vaccine protects against A (H1N1) pdm09 heterologous challenge without vaccine associated enhanced respiratory disease. Virology 2014, 471-473, 93-104.
18. Masic, A.; Lu, X.; Li, J.; Mutwiri, G. K.; Babiuk, L. A.; Brown, E. G.; Zhou, Y. Immunogenicity and protective efficacy of an elastase-dependent live attenuated swine influenza virus vaccine administered intranasally in pigs. Vaccine 2010, 28, 7098-7108.
19. Masic, A.; Booth, J. S.; Mutwiri, G. K.; Babiuk, L. A.; Zhou, Y. Elastase-dependent live attenuated swine influenza A viruses are immunogenic and confer protection against swine influenza A virus infection in pigs. J. Virol. 2009, 83, 10198-10210.
20. Richt, J. A.; Lekcharoensuk, P.; Lager, K. M.; Vincent, A. L.; Loiacono, C. M.; Janke, B. H.; Wu, W. H.; Yoon, K. J.; Webby, R. J.; Solorzano, A.; et al. Vaccination of pigs against swine influenza viruses by using an NS1-truncated modified live-virus vaccine. J. Virol. 2006, 80, 11009-11018.
21. Sharma, A.; Zeller, M. A.; Li, G.; Harmon, K. M.; Zhang, J.; Hoang, H.; Anderson, T. K.; Vincent, A. L.; Gauger, P. C. Detection of live attenuated influenza vaccine virus and evidence of reassortment in the U.S. swine population. J. Vet. Diagn. Investig. 2020, 32, 301-311.
Loeffen, W. L.; de Vries, R. P.; Stockhofe, N.; van Zoelen-Bos, D.; Maas, R.; Koch, G.; Moormann, R. J.; Rottier, P. J.; de Haan, C. A. Vaccination with a soluble recombinant hemagglutinin trimer protects pigs against a challenge with pandemic (H1N1) 2009 influenza virus. Vaccine 2011, 29, 1545-1550.
23. Rajao, D. S.; Loving, C. L.; Gauger, P. C.; Kitikoon, P.; Vincent, A. L. Influenza A virus hemagglutinin protein subunit vaccine elicits vaccine-associated enhanced respiratory disease in pigs. Vaccine 2014, 32, 5170-5176.
24. Bragstad, K.; Vinner, L.; Hansen, M. S.; Nielsen, J.; Fomsgaard, A. A polyvalent influenza A DNA vaccine induces heterologous immunity and protects pigs against pandemic A (H1N1) pdm09 virus infection. Vaccine 2013, 31, 2281-2288.
25. Gorres, J. P.; Lager, K. M.; Kong, W. P.; Royals, M.; Todd, J. P.; Vincent, A. L.; Wei, C. J.; Loving, C. L.; Zanella, E. L.; Janke, B.; et al. DNA Vaccination Elicits Protective Immune Responses against Pandemic and Classic Swine Influenza Viruses in Pigs. Clin. Vaccine Immunol. 2011, 18, 1987-1995.
26. Borggren, M.; Nielsen, J.; Karlsson, I.; Dalgaard, T. S.; Trebbien, R.; Williams, J. A.; Fomsgaard, A. A polyvalent influenza DNA vaccine applied by needle-free intradermal delivery induces cross-reactive humoral and cellular immune responses in pigs. Vaccine 2016, 34, 3634-3640.
27. Karlsson, I.; Borggren, M.; Rosenstierne, M. W.; Trebbien, R.; Williams, J. A.; Vidal, E.; Vergara-Alert, J.; Foz, D. S.; Darji, A.; Sistere-Oro, M.; et al. Protective effect of a polyvalent influenza DNA vaccine in pigs. Vet. Immunol. Immunopathol. 2018, 195, 25-32.
28. Macklin, M. D.; McCabe, D.; McGregor, M. W.; Neumann, V.; Meyer, T.; Callan, R.; Hinshaw, V. S.; Swain, W. F. Immunization of pigs with a particle-mediated DNA vaccine to influenza A virus protects against challenge with homologous virus. J. Virol. 1998, 72, 1491-1496.
29. Larsen, D. L.; Olsen, C. W. Effects of DNA dose, route of vaccination, and coadministration of porcine interleukin-6 DNA on results of DNA vaccination against influenza virus infection in pigs. Am. J. Vet. Res. 2002, 63, 653-659.
30. Erdman, M. M.; Kamrud, K. I.; Harris, D. L.; Smith, J. Alphavirus replicon particle vaccines developed for use in humans induce high levels of antibodies to influenza virus hemagglutinin in swine: Proof of concept. Vaccine 2010, 28, 594-596.
31. Demoulins, T.; Ruggli, N.; Gerber, M.; Thomann-Harwood, L. J.; Ebensen, T.; Schulze, K.; Guzman, C. A.; Mccullough, K. C. Self-Amplifying Pestivirus Replicon RNA Encoding Influenza Virus Nucleoprotein and Hemagglutinin Promote Humoral and Cellular Immune Responses in Pigs. Front. Immunol. 2020, 11, 622385.
32. Vander Veen, R. L.; Loynachan, A. T.; Mogler, M. A.; Russell, B. J.; Harris, D. L.; Kamrud, K. I. Safety, immunogenicity, and efficacy of an alphavirus replicon-based swine influenza virus hemagglutinin vaccine. Vaccine 2012, 30, 1944-1950.
33. do Nascimento, G. M.; Bugybayeva, D.; Patil, V.; Schrock, J.; Yadagiri, G.; Renukaradhya, G. J.; Diel, D. G. An Orf-Virus (ORFV)-Based Vector Expressing a Consensus H1 Hemagglutinin Provides Protection against Diverse Swine Influenza Viruses. Viruses 2023, 15, 994.
34. Joshi, L. R.; Knudsen, D.; Pineyro, P.; Dhakal, S.; Renukaradhya, G. J.; Diel, D. G. Protective Efficacy of an Orf Virus-Vector Encoding the Hemagglutinin and the Nucleoprotein of Influenza A Virus in Swine. Front. Immunol. 2021, 12, 747574.
35. Braucher, D. R.; Henningson, J. N.; Loving, C. L.; Vincent, A. L.; Kim, E.; Steitz, J.; Gambotto, A. A.; Kehrli, M. E., Jr. Intranasal vaccination with replication-defective adenovirus type 5 encoding influenza virus hemagglutinin elicits protective immunity to homologous challenge and partial protection to heterologous challenge in pigs. Clin. Vaccine Immunol. 2012, 19, 1722-1729.
36. Wesley, R. D.; Tang, M.; Lager, K. M. Protection of weaned pigs by vaccination with human adenovirus 5 recombinant viruses expressing the hemagglutinin and the nucleoprotein of H3N2 swine influenza virus. Vaccine 2004, 22, 3427-3434.
37. Wesley, R. D.; Lager, K. M. Overcoming maternal antibody interference by vaccination with human adenovirus 5 recombinant viruses expressing the hemagglutinin and the nucleoprotein of swine influenza virus. Vet. Microbiol. 2006, 118, 67-75.
38. Wesley, R. D.; Lager, K. M. Evaluation of a recombinant human adenovirus-5 vaccine administered via needle-free device and intramuscular injection for vaccination of pigs against swine influenza virus. Am. J. Vet. Res. 2005, 66, 1943-1947.
39. Klingbeil, K.; Lange, E.; Blohm, U.; Teifke, J. P.; Mettenleiter, T. C.; Fuchs, W. Protection of pigs against pandemic swine origin H1N1 influenza A virus infection by hemagglutinin—or neuraminidase-expressing attenuated pseudorabies virus recombinants. Virus Res. 2015, 199, 20-30.
40. Klingbeil, K.; Lange, E.; Teifke, J. P.; Mettenleiter, T. C.; Fuchs, W. Immunization of pigs with an attenuated pseudorabies virus recombinant expressing the haemagglutinin of pandemic swine origin H1N1 influenza A virus. J. Gen. Virol. 2014, 95, 948-959.
41. Dhanwani, R.; Zhou, Y.; Huang, Q.; Verma, V.; Dileepan, M.; Ly, H.; Liang, Y. A Novel Live Pichinde Virus-Based Vaccine Vector Induces Enhanced Humoral and Cellular Immunity after a Booster Dose. J. Virol. 2015, 90, 2551-2560.
42. Kumar, P.; Sharafeldin, T. A.; Kumar, R.; Huang, Q.; Liang, Y.; Goyal, S. M.; Porter, R. E.; Ly, H.; Mor, S. K. Development of a Recombinant Pichinde Virus-Vectored Vaccine against Turkey Arthritis Reovirus and Its Immunological Response Characterization in Vaccinated Animals. Pathogens 2021, 10, 197.
43. Kumari, S.; Chaudhari, J.; Huang, Q.; Gauger, P.; De Almeida, M. N.; Liang, Y.; Ly, H.; Vu, H. L. X. Immunogenicity and Protective Efficacy of a Recombinant Pichinde Viral-Vectored Vaccine Expressing Influenza Virus Hemagglutinin Antigen in Pigs. Vaccines 2022, 10, 1400.
44. WHO. Manual for the Laboratory Diagnosis and Virological Surveillance of Influenza; World Health Organization: Geneva, Switzerland, 2011; Chapter 2.E; pp. 43-57.
45. Halbur, P. G.; Paul, P. S.; Frey, M. L.; Landgraf, J.; Eernisse, K.; Meng, X. J.; Lum, M. A.; Andrews, J. J.; Rathje, J. A. Comparison of the pathogenicity of two US porcine reproductive and respiratory syndrome virus isolates with that of the Lelystad virus. Vet. Pathol. 1995, 32, 648-660.
46. van Oirschot, J. T. Diva vaccines that reduce virus transmission. J. Biotechnol. 1999, 73, 195-205.
47. van Oirschot, J. T.; Kaashoek, M. J.; Rijsewijk, F. A.; Stegeman, J. A. The use of marker vaccines in eradication of herpesviruses. J. Biotechnol. 1996, 44, 75-81.
48. Capua, I.; Schmitz, A.; Jestin, V.; Koch, G.; Marangon, S. Vaccination as a tool to combat introductions of notifiable avian influenza viruses in Europe, 2000 to 2006. Rev. Sci. Tech. 2009, 28, 245-259.

### Example 3

### Evaluation of Heterologous Protection

The hemagglutination inhibition (HI) assay is used as a model system to evaluate the homologous and heterologous protection of a SIV hemagglutinin expressed by the genetically modified Pichinde virus-based reverse genetics system described herein. After immunization, the HI assay is used to quantify the HI titer of the immunized swine serum samples. An HI titer threshold of 1:40 is generally recognized as providing 50% protection against the influenza virus; the HI titer of >40 is a useful and important benchmark for the qualitative assessment of protection (Reber and Katz, Expert Rev Vaccines. 2013 May; 12 (5): 519-536. doi: 10.1586/erv.13.35).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless SO specified.

1. A genetically engineered Pichinde virus comprising:
three ambisense genomic segments,
wherein the first genomic segment comprises a coding region encoding a Z protein and a coding region encoding an L RdRp protein,
wherein the second genomic segment comprises a coding region encoding a nucleoprotein (NP) and a second coding region, wherein the second coding region encodes at least one SIV protein, and
wherein the third genomic segment comprises a coding region encoding a glycoprotein and a third coding region, wherein the third coding region encodes at least one SIV protein.

2. The virus of claim 1 wherein the at least one SIV protein encoded by the second coding region is different than the at least one SIV protein encoded by the third coding region.

3. The virus of claim 1 wherein the at least one SIV protein encoded by the second coding region is the same as the at least one SIV protein encoded by the third coding region.

4. The virus of claim 1 wherein the second coding region, the third coding region, or both encode a polycistronic message encoding at least two SIV proteins.

5. The virus of claim 4 wherein the SIV proteins encoded by the second coding region are different than the SIV proteins encoded by the third coding region.

6. The virus of claim 4 wherein the SIV proteins encoded by the second coding region are the same as the SIV proteins encoded by the third coding region.

7. The virus of claim 1 wherein the second coding region, the third coding region, or both express a monocistronic message encoding at least two SIV proteins, wherein the monocistronic message comprises nucleotides encoding a self-cleaving peptide, and wherein the nucleotides are located between the SIV proteins.

8. The virus of claim 7 wherein the self-cleaving peptide comprises a 2A peptide.

9. The virus of claim 1 wherein the at least one SIV protein of the second genomic segment and the at least one SIV protein of the third genomic segment are selected from SIV hemagglutinin (HA) proteins.

10. The virus of claim 9 wherein the HA proteins are selected from a HA expressed by a SIV subtype H1N1, a HA expressed by a SIV subtype H1N2, or a HA expressed by a SIV subtype H3N2.

11. An infectious virus particle comprising the three genomic segments of claim 1.

12. A composition comprising an isolated infectious virus particle of claim 11.

13. A collection of vectors comprising:
a first vector encoding the first genomic segment of claim 1, wherein the first genomic segment is antigenomic,
a second vector encoding the second genomic segment of claim 1, wherein the second genomic segment is antigenomic, and
a third vector encoding the third genomic segment of claim 1, wherein the third genomic segment is antigenomic.

14. A method for making a genetically engineered Pichinde virus comprising:
introducing into a cell the collection of vectors of claim 13; and
incubating the cells in a medium under conditions suitable for expression and packaging of the first, second, and third genomic segments.

15. A reverse genetics system for making a genetically engineered virus comprising three vectors,
wherein a first vector encodes the first genomic segment of claim 1, wherein the first genomic segment is antigenomic,
wherein the second vector encodes the second genomic segment of claim 1, wherein the second genomic segment is antigenomic, and
wherein the third vector encodes the third genomic segment of claim 1, wherein the third genomic segment is antigenomic.

16. A method for using a reverse genetics system, comprising:
introducing into a cell the three vectors of genomic segments of claim 15; and
incubating the cell under conditions suitable for transcription of the three genomic segments and expression of the coding regions of each genomic segment.

17. A method for producing an immune response in a subject, comprising:
administering to a subject the infectious virus particle of claim 11.

18. The method of claim 17 wherein the subject is a porcine animal.

19. The method of claim 17 wherein the immune response comprises a humoral immune response.

20. The method of claim 17 wherein the immune response comprises a cell-mediated immune response.
